(19) United States
(12) Patent Application Publication
(10) Pub. No.: US 2022/0057415 A1
(43) Pub. Date: Feb. 24, 2022

(54) METHOD FOR DETECTING ALLERGIC DISEASES

(71) Applicant: The University of Tokyo, Tokyo (JP)

(72) Inventors: Takahisa MURATA, Tokyo (JP); Tatsuro NAKAMURA, Tokyo (JP); Yuri TACHIBANA, Tokyo (JP); Akane HAYASHI, Tokyo (JP)

(73) Assignee: The University of Tokyo, Tokyo (JP)

(21) Appl. No.: 16/978,291

(22) PCT Filed: Mar. 11, 2019
(86) PCT No.: PCT/JP2019/009835

§ 371 Date: Nov. 25, 2020

(30) Foreign Application Priority Data
Mar. 9, 2018 (JP) 2018-043404

(51) Int. Cl. G01N 33/92; G01N 33/50

(52) U.S. Cl. CPC G01N 33/92; G01N 2800/24; G01N 33/5038

(57) ABSTRACT

An object of the present invention is to provide a novel method for detecting an allergic disease (for example, allergic rhinitis or allergic conjunctivitis) and an activation state of mast cells and/or eosinophils, and a novel method for determining a therapeutic effect on an allergic disease. The present invention provides a method for detecting an allergic disease or an activation state of mast cells and/or eosinophils in the nasal cavities or eyes, comprising the step of measuring the concentration of a lipid metabolite in a biological sample of a subject. The present invention also provides a method for determining a therapeutic effect on an allergic disease, comprising the step of measuring the concentration of a lipid metabolite in a biological sample of a subject.

### CROSS-REFERENCE TO RELATED APPLICATION

The present application enjoys the benefit of priority from the prior Japanese Patent Application No. 2018-43403 filed on Mar. 9,2018, the entire disclosure of which is incorporated herein by reference.

### TECHNICAL FIELD

The present invention relates to a method for detecting an allergic disease. The present invention also relates to a method for detecting an activation state of mast cells and/or eosinophils in the nasal cavities or eyes, and a method for determining a therapeutic effect on an allergic disease.

### BACKGROUND ART

The number of patients with allergic rhinitis, among allergic diseases, is increasing worldwide year by year, and 40% or more of the people in Japan suffer from allergic rhinitis (Ministry of Health, Labor and Welfare, 2016). Allergic rhinitis is caused by the uptake of an antigen such as pollen or house dust into the body, and accompanied by symptoms such as sneezing, rhinorrhea and nasal congestion. General malaise and sleep disorder caused by these symptoms significantly reduce the quality of life (QOL) of patients with allergic rhinitis.

As methods for diagnosis of allergic rhinitis, measurement of the blood IgE concentration, intradermal tests, scratch tests, measurement of the number of eosinophils in nasal mucus, and the like are performed. However, these diagnostic methods involve problems that they may not correlate with the progress of disease state (symptoms) and are invasive, and that it is difficult for them to distinguish allergic rhinitis from diseases such as upper respiratory inflammation (cold) and asthma (Non-Patent Document 1). Under such a background, reaction inducing tests using an antigen are conducted as definitive diagnosis of allergic rhinitis, but impose a heavy burden on patients, and, further, there is a problem that such diagnosis can be conducted only in a limited number of hospitals.

### REFERENCE LIST

### Non-Patent Document

Non-Patent Document 1: Chawes, B. L. K., Upper and lower airway pathology in young children with allergic- and non-allergic rhinitis, Danish Medical Journal 1-23 (2011)

### SUMMARY OF THE INVENTION

An object of the present invention is to provide a novel method for detecting an allergic disease. Another object of the present invention is to provide a novel method for detecting an activation state of mast cells and/or eosinophils in the nasal cavities or eyes, and a novel method for determining a therapeutic effect on an allergic disease.

The present inventors have confirmed the excretion of a lipid metabolite specific to allergic rhinitis in nasal secretions of allergic rhinitis model mice in which activation of mast cells and eosinophils is observed in the nasal cavities. Further, the present inventors have found that the concentration of a specific lipid metabolite in nasal secretions can be used as an index to detect allergic rhinitis and an activation state of mast cells and eosinophils in the nasal cavities. Furthermore, the present inventors have confirmed the excretion of a lipid metabolite specific to allergic conjunctivitis in lacrimal fluids secreted from the eyes of allergic conjunctivitis model guinea pigs in which activation of mast cells and eosinophils is observed. The present inventors have also found that the concentration of a specific lipid metabolite in eye secretions can be used as an index to detect allergic conjunctivitis and an activation state of mast cells and eosinophils in the eyes. The present invention is based on these findings.

The present invention provides the following inventions.

[1] A method for detecting an allergic disease or an activation state of mast cells and/or eosinophils in the nasal cavities or eyes, comprising the step of measuring the concentration of a lipid metabolite in a biological sample of a subject.

[2] The method for detection according to [1], wherein, when the concentration of the lipid metabolite in the biological sample of the subject is above the concentration of the lipid metabolite in a biological sample of a healthy subject, it is indicated that the subject suffers from an allergic disease or that mast cells and/or eosinophils are activated in the nasal cavities or eyes of the subject.

[3] A method for determining a therapeutic effect on an allergic disease, comprising the step of measuring the concentration of a lipid metabolite in a biological sample of a subject.

[4] The method for determination according to [3], wherein, when the concentration of the lipid metabolite in the biological sample of the subject is below the concentration of the lipid metabolite in the biological sample of the subject before treatment or the concentration of the lipid metabolite in a biological sample of a subject suffering from an allergic disease, it is indicated that the treatment has a therapeutic effect.

[5] The method for detection according to [1] or [2], or the method for determination according to [3] or [4], wherein the allergic disease is allergic rhinitis or allergic conjunctivitis.

[6] The method for determination according to any one of [3] to [5], wherein the allergic disease is allergic rhinitis, and wherein the treatment of allergic rhinitis is drug therapy, immunotherapy or surgical treatment.

[7] The method for detection according to [5], or the method for determination according to [5] or [6], wherein the allergic rhinitis is seasonal allergic rhinitis or perennial allergic rhinitis.

[8] The method for detection according to any one of [1], [2], [5] and [7], or the method for determination according to any one of [3] to [7], wherein the biological sample is a body fluid noninvasively collected from the subject.

[9] The method for detection according to any one of [1], [2], [5], [7] and [8], or the method for determination according to any one of [3] to [8], wherein the lipid metabolite is one or more lipid metabolite(s) selected from the group consisting of an arachidonic acid metabolite, an eicosapentaenoic acid metabolite, a docosahexaenoic acid metabolite, a linolenic acid metabolite and an eicosadienoic acid metabolite.

[10] The method for detection according to any one of [1], [2], [5] and [7] to [9], or the method for determination according to any one of [3] to [9], wherein the lipid metabolite is one or more lipid metabolite(s) selected from the group consisting of PGD₂, PGE₂, 12-HETE, 15-HETE, 12-HEPE, 15-HEPE, 14-HDOHE, 8-HDOHE, 13-HODE, 8-isoPGE₂, 16-HDoHE, 15-HEDE, 15-OxoEDE, 20-HETE, 14,15-EET, 13,14-dihydro-15-keto-PGD₂, PGJ₂, LTB₄, 9-HOTrE, 13-HOTrE, 9,10-DiHOME, 12,13-DiHOME, 13-HpODE, 18-HEPE, 10-HDoHE and 20-HDoHE.

[11] The method for detection according to any one of [1], [2], [5] and [7] to [10], or the method for determination according to any one of [3] to [10], wherein the concentration of the lipid metabolite is measured by mass spectrometry.

[12] Use of a lipid metabolite as an allergic disease marker or an activation marker for mast cells and/or eosinophils in the nasal cavities or eyes.

[13] The use according to [12], wherein the allergic disease is allergic rhinitis or allergic conjunctivitis.

[14] The use according to [12] or [13], wherein the allergic disease is allergic rhinitis, and wherein the allergic rhinitis is seasonal allergic rhinitis or perennial allergic rhinitis.

[15] The use according to any one of [12] to [14], wherein the lipid metabolite is one or more lipid metabolite(s) selected from the group consisting of an arachidonic acid metabolite, an eicosapentaenoic acid metabolite, a docosahexaenoic acid metabolite, a linolenic acid metabolite and an eicosadienoic acid metabolite.

[16] The use according to any one of [12] to [15], wherein the lipid metabolite is one or more lipid metabolite(s) selected from the group consisting of PGD₂, PGE₂, 12-HETE, 15-HETE, 12-HEPE, 15-HEPE, 14-HDoHE, 8-HDoHE, 13-HODE, 8-isoPGE₂, 16-HDoHE, 15-HEDE, 15-OxoEDE, 20-HETE, 14,15-EET, 13,14-dihydro-15-keto-PGD₂, PGJ₂, LTB₄, 9-HOTrE, 13-HOTrE, 9,10-DiHOME, 12,13-DiHOME, 13-HpODE, 18-HEPE, 10-HDoHE and 20-HDoHE.

[17] A method for screening for a therapeutic drug for an allergic disease, comprising the steps of administering a candidate drug for a therapeutic drug for an allergic disease to a subject; and measuring the concentration of a lipid metabolite in a biological sample of the subject.

[18] The method for screening according to [17], wherein, when the concentration of the lipid metabolite in the biological sample of the subject after the administration of the candidate drug is below the concentration of the lipid metabolite in the biological sample of the subject before the administration of the candidate drug or the concentration of the lipid metabolite in a biological sample of a subject suffering from the allergic disease, it is indicated that the therapeutic drug has a therapeutic effect.

[19] A method for identifying, in a lipid metabolite in a biological sample, an allergic disease marker or an activation marker for mast cells and/or eosinophils in the nasal cavities or eyes, comprising the steps of measuring the concentration of a lipid metabolite in a biological sample of a subject suffering from an allergic disease and the concentration of the lipid metabolite in a biological sample of a healthy subject; and comparing the measured two concentrations.

[20] The method for identification according to [19], wherein, when the concentration of the lipid metabolite in the biological sample of the subject suffering from the allergic disease is above the concentration of the lipid metabolite in the biological sample of the healthy subject, it is indicated that the lipid metabolite serves as an allergic disease marker or an activation marker for mast cells and/or eosinophils in the nasal cavities or eyes.

[21] The method for screening according to [17] or [18], or the method for identification according to [19] or [20], wherein the allergic disease is allergic rhinitis or allergic conjunctivitis.

The present invention is advantageous in enabling simple and accurate detection of an allergic disease and a therapeutic effect on the allergic disease.

### BRIEF DESCRIPTION OF THE DRAWINGS

FIG. 1 is a graph showing the ovalbumin-specific IgE levels in the sera of an unsensitized group and an OVA-sensitized group sensitized upon administration of OVA. * denotes P<0.05.

FIG. 2 is a graph showing a transition of the number of sneezes for 10 minutes after the administration of a solvent or OVA ** denotes P<0.01 vs solvent administered group.

FIG. 3 is a graph showing a transition of the number of sneezes in a solvent-administered group or an upper respiratory inflammation model group to which LPS was administered (LPS-administered group). ** denotes P<0.01 vs single-time solvent-administered group.

FIG. 4 is a graph showing a change in nasal cavity volume before the administration of the solvent or OVA, 24 hours after the fifth administration, and 24 hours after the tenth administration. * denotes P<0.05.

FIG. 5 shows a typical example of an HE-stained image of a cross section of the nose. The enlarged view on the right shows a nasal turbinate.

FIG. 6 is a graph showing quantification results of the number of mucosal type mast cells infiltrated into nasal turbinates (nasal cavities) in allergic rhinitis model mice to which OVA was administered, upper respiratory inflammation model mice to which LPS was administered, and control groups. ** denotes P<0.01 vs 10-time solvent-administered group.

FIG. 7 is a graph showing quantification results of the number of connective tissue type mast cells infiltrated into nasal turbinates in the allergic rhinitis model mice, the upper respiratory inflammation model mice, and the control groups.

FIG. 8 is a graph showing quantification results of the number of eosinophils infiltrated into nasal turbinates in the allergic rhinitis model mice, the upper respiratory inflammation model mice, and the control groups. ** denotes P<0.01 vs 10-time solvent-administered group. # denotes P<0.01 vs 5-time solvent-administered group. $ denotes P<0.01 vs LPS-administered group.

FIG. 9 is a graph showing quantification results of the number of neutrophils infiltrated into nasal turbinates in the allergic rhinitis model mice, the upper respiratory inflammation model mice, and the control groups. * denotes P<0.05 vs single-time solvent-administered group.

FIG. 10 includes graphs showing relative values of lipid mediators (A: PGD₂, B: PGE₂) at the downstream of COX (derived from AA), which were detected in nasal lavage fluids of the allergic rhinitis model mice, the upper respiratory inflammation model mice, and the control groups. * denotes P<0.05 vs 10-time solvent-administered group. # denotes P<0.05 vs 5-time solvent-administered group or P<0.05 vs LPS-administered group.

FIG. 11 includes graphs showing relative values of lipid mediators (A: 12-HETE, B:15-HETE) at the downstream of 12/15-LOX (derived from AA), which were detected in nasal lavage fluids of the allergic rhinitis model mice, the upper respiratory inflammation model mice, and the control groups. ** denotes P<0.01 vs 10-time solvent-administered group. * denotes P<0.05 vs 5-time or 10-time solvent-administered group.

FIG. 12 includes graphs showing relative values of lipid mediators (A: 12-HEPE, B: 15-HEPE) at the downstream of 12/15-LOX (derived from EPA), which were detected in nasal lavage fluids of the allergic rhinitis model mice, the upper respiratory inflammation model mice, and the control groups. * denotes P<0.05 vs 5-time or 10-time solvent-administered group.

FIG. 13 includes graphs showing relative values of a lipid mediator (A: 14-HDoHE) at the downstream of 12/15-LOX (derived from DHA) and a lipid mediator (B: 8-HDoHE) at the downstream of 5-LOX (derived from DHA), which were detected in nasal lavage fluids of the allergic rhinitis model mice, the upper respiratory inflammation model mice, and the control groups. * denotes P<0.05 vs 5-time solvent-administered group. ** denotes P<0.01 vs 10-time solvent-administered group.

FIG. 14 is a graph showing relative values of a lipid mediator (13-HODE) at the downstream of 12/15-LOX (derived from LA), which was detected in nasal lavage fluids of the allergic rhinitis model mice, the upper respiratory inflammation model mice, and the control groups. * denotes P<0.05 vs single-time solvent-administered group. # denotes P<0.05 vs LPS-administered group.

FIG. 15 is a graph showing relative values of a lipid mediator (8-isoPGE₂) under OX (derived from AA), which was detected in nasal lavage fluids of the allergic rhinitis model mice, the upper respiratory inflammation model mice, and the control groups. * denotes P<0.05 vs 10-time solvent-administered group.

FIG. 16 is a graph showing relative values of a lipid mediator (16-HDoHE) under OX (derived from DHA), which was detected in nasal lavage fluids of the allergic rhinitis model mice, the upper respiratory inflammation model mice, and the control groups.

FIG. 17 includes graphs showing relative values of a lipid mediator (A: 15-HEDE) under OX (derived from EDA) and a metabolite of 15-HEDE (B: 15-OxoEDE), which were detected in nasal lavage fluids of the allergic rhinitis model mice, the upper respiratory inflammation model mice, and the control groups. * denotes P<0.05 vs 10-time solvent-administered group.

FIG. 18 includes graphs showing relative values of lipid mediators (A: 20-HETE, B: 14,15-EET) at the downstream of CYP (derived from AA), which were detected in nasal lavage fluids of the allergic rhinitis model mice, the upper respiratory inflammation model mice, and the control groups. * denotes P<0.05 vs 10-time solvent-administered group.

FIG. 19 is a graph showing relative values of a lipid mediator (PGD₂) under COX (derived from AA), which was detected in urine of the allergic rhinitis model mice and the control groups.

FIG. 20 is a graph showing relative values of a lipid mediator (12-HETE) under 12/15-LOX (derived from AA), which was detected in the urine of the allergic rhinitis model mice and the control groups.

FIG. 21 is a graph showing relative values of a lipid mediator (12-HEPE) under 12/15-LOX (derived from EPA) detected in the urine of the allergic rhinitis model mice and the control groups.

FIG. 22 shows swelling of the conjunctivae of an unsensitized guinea pig, an allergic conjunctivitis model guinea pig, and an infectious conjunctivitis model guinea pig.

FIG. 23 shows the conjunctivae of the solvent-administered group, the allergic conjunctivitis model guinea pig and the infectious conjunctivitis model guinea pig.

FIG. 24 includes graphs showing the vasopermeability, water content and thickness of the conjunctiva of the solvent-administered group, an allergic conjunctivitis group, and an infectious conjunctivitis group.

FIG. 25 shows typical examples of an HE-stained image and a May-Grünwald Giemsa-stained image of cross sections of the conjunctivae of the allergic conjunctivitis group and the infectious conjunctivitis group.

FIG. 26 includes graphs showing relative values of lipid mediators (PGD₂, 13,14-dihydro-15-keto-PGD₂ and PGJ₂) which were detected in lacrimal fluids and conjunctival sac lavage fluids of an unsensitized group, the allergic conjunctivitis group and the infectious conjunctivitis group. 13,14-dihydro-15-keto-PGD₂ is 9α-hydroxy-11,15-dioxo-prost-5Z-en-1-oic acid. PGJ₂ is 11-oxo-15S-hydroxy-prosta-5Z,9,13E-trien-1-oic acid.

FIG. 27 includes graphs showing relative values of lipid mediators (PGE₂, 8-iso-PGE₂ and 12-KETE) which were detected in lacrimal fluids and conjunctival sac lavage fluids of the unsensitized group, the allergic conjunctivitis group and the infectious conjunctivitis group. PGE₂ is 9-oxo-11α,15S-dihydroxy-prosta-5Z,13E-dien-1-oic acid. 8-iso-PGE₂ is 9-oxo-11α, 15S-dihydroxy-(8β)-prosta-5Z,13E-dien-1-oic acid. 12-KETE is 12-oxo-5Z,8Z,10E,14Z-eicosatetraenoic acid.

FIG. 28 includes graphs showing relative values of lipid mediators (LTB₄, 20-HETE, 15-HETE and 12-HETE) which were detected in lacrimal fluids and conjunctival sac lavage fluids of the unsensitized group, the allergic conjunctivitis group and the infectious conjunctivitis group. LTB₄ is 5S,12R-dihydroxy-6Z,8E,10E,14Z-eicosatetraenoic acid.

FIG. 29 includes graphs showing relative values of lipid mediators (9-HOTrE, 13-HOTrE, 9,10-DiHOME and 12,13-DiHOME) which were detected in lacrimal fluids and conjunctival sac lavage fluids of the unsensitized group, the allergic conjunctivitis group and the infectious conjunctivitis group. 9-HOTrE is 9S-hydroxy-10E,12Z,15Z-octadecatrienoic acid. 13-HOTrE is 13S-hydroxy-9Z,11E,15Z-octadecatrienoic acid. 9,10-DiHOME is (±) 9,10-dihydroxy-12Z-octadecenoic acid. 12,13-DiHOME is (±) 12,13-dihydroxy-9Z-octadecenoic acid.

FIG. 30 includes graphs showing relative values of lipid mediators (13-HpODE, 18-HETE and 12-HEPE) which were detected in lacrimal fluids and conjunctival sac lavage fluids of the unsensitized group, the allergic conjunctivitis group and the infectious conjunctivitis group. 13-HpODE is (±) 13-hydroperoxy-9Z,11E-octadecadienoic acid. 18-HEPE is (±) 18-hydroxy-5Z,8Z,11Z,14Z,16E-eicosapentaenoic acid.

FIG. 31 includes graphs showing relative values of lipid mediators (8-HDoHE, 10-HDoHE, 16-HDoHE and 20-HDoHE) which were detected in lacrimal fluids and conjunctival sac lavage fluids of the unsensitized group, the allergic conjunctivitis group and the infectious conjunctivitis group. 10-HDoHE is (±) 10-hydroxy-4Z,7Z,11E,13Z,16Z,19Z-docosahexaenoic acid. 20-HDoHE is (±) 20-hydroxy-4Z,7Z,10Z,13Z,16Z,18E-docosahexaenoic acid.

### DETAILED DESCRIPTION OF THE INVENTION

In the present invention, the term “lipid metabolite” means a lipid degradation product produced in vivo by degradation caused by enzyme-dependent oxidation or enzyme-independent oxidation (sometimes abbreviated as “OX” herein), and includes lipid mediators having a physiological effect of controlling inflammatory reactions. Enzyme-dependent oxidation progresses by a lipid metabolizing enzyme present in vivo. The enzyme includes lipid metabolizing enzymes involved in onset and development of allergic rhinitis (preferably, lipid metabolizing enzymes activated by the onset and development of allergic rhinitis), such as cyclooxygenase (sometimes abbreviated as “COX” herein), lipoxygenase (sometimes abbreviated as “LOX” herein) (for example, 12/15LOX and 5-LOX), enzyme-independent oxidation, and cytochrome p450 (sometimes abbreviated as “CYP” herein). Examples of the lipid that is degraded by enzyme-dependent oxidation or enzyme-independent oxidation include arachidonic acid, eicosapentaenoic acid, docosahexaenoic acid, linolenic acid and eicosadienoic acid.

Examples of the lipid metabolite in the present invention include arachidonic acid metabolites, eicosapentaenoic acid metabolites, docosahexaenoic acid metabolites, linolenic acid metabolites and eicosadienoic acid metabolites. Preferred examples of the lipid metabolite include COX metabolites of arachidonic acid (e.g., PGD₂ and PGE₂), LOX metabolites of arachidonic acid (e.g., 12/15LOX metabolites of arachidonic acid, such as 12-HETE and 15-HETE), OX metabolites of arachidonic acid (e.g., 8-isoPGE₂), CYP metabolites of arachidonic acid (e.g., 20-HETE and 14,15-EET), LOX metabolites of eicosapentaenoic acid (e.g., 12/15LOX metabolites of eicosapentaenoic acid, such as 12-HEPE and 15-HEPE), LOX metabolites of docosahexaenoic acid (e.g., 12/15LOX metabolites of docosahexaenoic acid such as 14-HDoHE, and 5-LOX metabolites of docosahexaenoic acid such as 8-HDoHE), OX metabolites of docosahexaenoic acid (e.g., 16-HDoHE), LOX metabolites of linolenic acid (e.g., 12/15LOX metabolites of linolenic acid, such as 13-HODE), and OX metabolites of eicosadienoic acid (e.g., 15-HEDE and 15-OxoEDE).

Lipid metabolites useful for detection of allergic rhinitis, detection of an activation state of mast cells and/or eosinophils in the nasal cavities or determination of a therapeutic effect on allergic rhinitis, as an allergic rhinitis marker or an activation marker for mast cells and/or eosinophils in the nasal cavities, or the like in the present invention are PGD₂, PGE₂, 12-HETE, 15-HETE, 12-HEPE, 15-HEPE, 14-HDOHE, 8-HDoHE, 13-HODE, 8-isoPGE₂, 16-HDoHE, 15-HEDE, 15-OxoEDE, 20-HETE, and 14,15-EET.

Lipid metabolites useful for detection of allergic conjunctivitis, detection of an activation state of mast cells and/or eosinophils in the eyes or determination of a therapeutic effect on allergic conjunctivitis, as an allergic conjunctivitis marker or an activation marker for mast cells and/or eosinophils in the eyes, or the like in the present invention are PGD₂, 13,14-dihydro-15-keto-PGD₂, PGJ₂, LTB₄, 20-HETE, 15-HETE, 12-HETE, 9-HOTrE, 13-HOTrE, 9,10-DiHOME, 12,13-DiHOME, 13-HpODE, 18-HEPE, 12-HEPE, 8-HDoHE, 10-HDoHE, 16-HDoHE and 20-HDoHE.

In the present invention, the “allergic disease” is a disease accompanied by an allergic reaction, and examples thereof include allergic rhinitis and allergic conjunctivitis.

In the present invention, the term “allergic rhinitis” means rhinitis caused by antigen stimulation, and includes seasonal allergic rhinitis and perennial allelic rhinitis. The seasonal allergic rhinitis, which is referred to also as pollinosis, is allergic rhinitis caused by pollen of Japanese cedar (sugi), Japanese cypress (hinoki), rice, Japanese white birch, ragweed, mugwort and Japanese hop. The perennial allergic rhinitis is allergic rhinitis caused by allergens such as house dust, mites, molds and pet hair.

In the present invention, the term “allergic conjunctivitis” means conjunctivitis caused by antigen stimulation, and includes seasonal allergic conjunctivitis and perennial allergic conjunctivitis. The seasonal allergic conjunctivitis is an allergic disease caused by pollen of Japanese cedar (sugi), Japanese cypress (hinoki), rice, Japanese white birch, ragweed, mugwort and Japanese hop. The perennial allergic conjunctivitis is an allergic conjunctivitis caused by allergens such as house dust, mites, molds and pet hair.

In the present invention, the term “biological sample” means a sample separated from a living body, and preferably represents a body fluid collected noninvasively from the living body (for example, nasal secretions, oral lavage fluids, secretions from the eyes such as lacrimal fluid, and urine).

In the present invention, the term “subject” is used with the meaning including not only humans, but also mammals other than humans (for example, monkeys, mice, rats, dogs, cats, rabbits, horses, cows, pigs and sheep).

According to a first aspect of the present invention, there is provided a method for detecting an allergic disease and a method for detecting an activation state of mast cells and/or eosinophils in the nasal cavities or eyes. The method for detection of the present invention can be used to detect an allergic disease and an activation state of mast cells and/or eosinophils in the nasal cavities or eyes (especially, an activation state of mast cells in the nasal cavities or eyes) using a lipid metabolite in a biological sample.

A preferred embodiment of the first aspect of the present invention provides a method for detecting allergic rhinitis and a method for detecting an activation state of mast cells and/or eosinophils in the nasal cavities. The method for detection of the present invention can be used to detect allergic rhinitis and an activation state of mast cells and/or eosinophils in the nasal cavities (especially, an activation state of mast cells in the nasal cavities) using a lipid metabolite in a biological sample.

In the method for detection according to the present invention, first, the step (A) of measuring the concentration of a lipid metabolite in a biological sample of a subject is carried out. The concentration of the lipid metabolite can be measured by a known method. For example, the concentration of the lipid metabolite can be measured by mass spectrometry, an ELISA method and an immunoassay such as an immunochromatography method. Examples of the mass spectrometry include liquid chromatography-mass spectrometry (LC-MS), liquid chromatography-tandem mass spectrometry (LC-MSMS), high performance liquid chromatography-mass spectrometry (HPLC-MS), and high-performance liquid chromatography-tandem mass spectrometry (HPLC-MSMS). The immunoassay is an analytical method using a detectably-labeled anti-lipid metabolite antibody or a detectably-labeled antibody (secondary antibody) against an anti-lipid metabolite antibody. Depending on the antibody labeling method, the immunoassays are classified into enzyme immunoassay (EIA or ELISA), radioimmunoassay (RIA), fluorescence immunoassay (FIA), a fluorescence polarization immunoassay (FPIA), chemiluminescence immunoassay (CLIA), and the like, all of which can be used in the present invention. From the viewpoint of accurately measuring the concentrations of lipid metabolites having similar structures, measurement by mass spectrometry (especially, LC-MSMS and HPLC-MSMS) is preferred.

In the method for detection of the present invention, the step of determining, in the subject from whom/which the biological sample has been collected, the presence or absence of allergic rhinitis or an activation state of either or both of mast cells and eosinophils in the nasal cavities based on the concentration of the lipid metabolite measured in the step (A) can further be carried out. In this step, when the concentration of the lipid metabolite in the biological sample of the subject is above the concentration of the lipid metabolite in a biological sample of a healthy subject (preferably, above with a significant difference), it is indicated that the subject suffers from allergic rhinitis or that mast cells and/or eosinophils are activated in the nasal cavities of the subject. That is, the method for detection of the present invention may further comprise the step (B1) of determining that the subject suffers from allergic rhinitis or that mast cells and/or eosinophils are activated in the nasal cavities of the subject, when the concentration of the lipid metabolite in the biological sample of the subject is above the concentration of the lipid metabolite in a biological sample of a healthy subject (preferably, above with a significant difference). As the concentration of the lipid metabolite in the biological sample of the healthy subject, there can be used a mean value calculated by collecting biological samples from a plurality of healthy subjects in advance and measuring the concentrations of the lipid metabolite in the biological samples. The phrase “suffers from allergic rhinitis” is used with the meaning including also the case where there is a possibility that the subject may suffer from allergic rhinitis, and the sentence that “mast cells and/or eosinophils are activated in the nasal cavities” means that these cells release an allergy-inducing substance such as histamine, which induces an allergic reaction, and is used with the meaning including also increases in numbers of mast cells and eosinophils and an increase in allergic reaction caused thereby. In the present invention, the “healthy subject” may be a subject who/which does not develop allergic rhinitis. For example, when the allergic rhinitis is seasonal allergic rhinitis caused by Japanese cedar pollen, the subject before dispersion of Japanese cedar pollen can be defined as the “healthy subject.”

According to the method for detection of the present invention, allergic rhinitis can be detected in the subject. Therefore, the method for detection of the present invention can be used as an auxiliary method in the diagnosis of allergic rhinitis, and, finally, a doctor or veterinarian can decide whether or not the subject suffers from allergic rhinitis, in combination with other findings in some cases.

The method for detection of the present invention can be used to detect allergic rhinitis and an activation state of mast cells and/or eosinophils in the nasal cavities based on the biological sample collected noninvasively from the subject. In other words, the method for detection of the present invention is advantageous in its ability to simply and accurately detect allergic rhinitis and an activation state of mast cells and/or eosinophils in the nasal cavities, while reducing the burden on the patient.

A preferred embodiment of the first aspect of the present invention also provides a method for detecting allergic conjunctivitis and a method for detecting an activation state of mast cells and/or eosinophils in the eyes. The method for detection of the present invention can be used to detect allergic conjunctivitis and an activation state of mast cells and/or eosinophils in the eyes (especially, an activation state of mast cells in the eyes) using a lipid metabolite in a biological sample.

In the method for detection according to the present invention, first, the step (A) of measuring the concentration of a lipid metabolite in a biological sample of a subject is carried out. The concentration of the lipid metabolite may be measured by a known method, as described above.

In the method for detection of the present invention, the step of determining, in the subject from whom/which the biological sample has been collected, the presence or absence of allergic conjunctivitis or an activation state of either or both of mast cells and eosinophils in the eyes based on the concentration of the lipid metabolite measured in the step (A) can further be carried out. In this step, when the concentration of the lipid metabolite in the biological sample of the subject is above the concentration of the lipid metabolite in a biological sample of a healthy subject (preferably, above with a significant difference), it is indicated that the subject suffers from allergic conjunctivitis or that mast cells and/or eosinophils are activated in the eyes of the subject. That is, the method for detection of the present invention may further comprise the step (B2) of determining that the subject suffers from allergic conjunctivitis or that mast cells and/or eosinophils are activated in the eyes of the subject, when the concentration of the lipid metabolite in the biological sample of the subject is above the concentration of the lipid metabolite in a biological sample of a healthy subject (preferably, above with a significant difference). As the concentration of the lipid metabolite in the biological sample of the healthy subject, there can be used a mean value calculated by collecting biological samples from a plurality of healthy subjects in advance and measuring the concentrations of the lipid metabolite in the biological samples. The phrase “suffers from allergic conjunctivitis” is used with the meaning including also the case where there is a possibility that the subject may suffer from allergic conjunctivitis, and the sentence that “mast cells and/or eosinophils are activated in the eyes” means that these cells release an allergy-inducing substance such as histamine, which induces an allergic reaction, and is used with the meaning including also increases in numbers of mast cells and eosinophils and an increase in allergic reaction caused thereby. In the present invention, the “healthy subject” may be a subject who/which does not develop allergic conjunctivitis. For example, when the allergic conjunctivitis is seasonal allergic conjunctivitis caused by Japanese cedar pollen, the subject before dispersion of Japanese cedar pollen can be defined as the “healthy subject.”

According to the method for detection of the present invention, allergic conjunctivitis can be detected in the subject. Therefore, the method for detection of the present invention can be used as an auxiliary method in the diagnosis of allergic conjunctivitis, and, finally, a doctor or veterinarian can decide whether or not the subject suffers from allergic conjunctivitis, in combination with other findings in some cases.

The method for detection of the present invention can be used to detect allergic conjunctivitis and an activation state of mast cells and/or eosinophils in the eyes based on the biological sample collected noninvasively from the subject. In other words, the method for detection of the present invention is advantageous in its ability to simply and accurately detect allergic conjunctivitis and an activation state of mast cells and/or eosinophils in the eyes, while reducing the burden on the patient.

According to a second aspect of the present invention, there is provided a method for determining a therapeutic effect on an allergic disease. The method for determination according to the present invention can be used to determine a therapeutic effect on an allergic disease using a lipid metabolite in a biological sample as an index.

A preferred embodiment of the second aspect of the present invention provides a method for determining a therapeutic effect on allergic rhinitis. The method for determination according to the present invention can be used to determine a therapeutic effect on allergic rhinitis using a lipid metabolite in a biological sample as an index.

In the method for determination according to the present invention, the step (C) of measuring the concentration of a lipid metabolite in a biological sample of a subject is carried out in a similar manner as in the method for detection of the present invention. The concentration of the lipid metabolite can be measured in a similar manner as in the method for detection of the present invention.

In the method for determination of the present invention, the step of determining a therapeutic effect on allergic rhinitis in the subject having undergone treatment based on the concentration of the lipid metabolite measured in the step (C) can further be carried out. In this step, when the concentration of the lipid metabolite in the biological sample of the subject having undergone treatment of allergic rhinitis is below the concentration of the lipid metabolite in the biological sample of the subject before the treatment or the concentration of the lipid metabolite in a biological sample of a subject suffering from allergic rhinitis (preferably, below with a significant difference), it is indicated that the treatment has a therapeutic effect. That is, the method for determination of the present invention may further comprise the step (D1) of determining that treatment of allergic rhinitis has a therapeutic effect, when the concentration of the lipid metabolite in the biological sample of the subject having undergone the treatment is below the concentration of the lipid metabolite in the biological sample of the subject before the treatment or the concentration of the lipid metabolite in a biological sample of a subject suffering from allergic rhinitis (preferably, below with a significant difference). As the concentration of the lipid metabolite in the biological sample of the subject before the treatment, there can be used a value obtained by measuring the concentration of the lipid metabolite in the biological sample of the subject before the treatment. As the concentration of the lipid metabolite in the biological sample of the healthy subject, there can be used a mean value calculated by collecting biological samples from a plurality of healthy subjects in advance and measuring the concentrations of the lipid metabolite in the biological samples. The subject who/which undergoes the treatment in the method for determination of the present invention is preferably a subject suffering from allergic rhinitis. In the present invention, the “subject suffering from allergic rhinitis” may be a subject demonstrated to have allergic rhinitis from results of other inspection methods, and can preferably be a subject diagnosed as developing allergic rhinitis by a doctor or veterinarian.

Examples of the treatment of allergic rhinitis on which a therapeutic effect can be determined by the method for determination of the present invention include drug therapy, immunotherapy and surgical treatment. The drug therapy includes treatment with therapeutic drugs for allergic rhinitis, and examples of such therapeutic drugs include antihistamines, leukotriene receptor antagonists and thromboxane A₂ inhibitors. The immunotherapy includes administration of allergens, and examples of such allergens include pollen such as Japanese cedar pollen. Examples of administration forms include sublingual administration, subcutaneous administration and translymph node administration.

The method for determination according to the present invention can be used to determine the therapeutic effect in the subject having undergone treatment of allergic rhinitis, thereby verifying the effectiveness of the treatment of allergic rhinitis performed on the subject. Then, if no therapeutic effect is observed, the treatment can be immediately stopped and another treatment plan can be made. Therefore, the method for determination of the present invention is advantageous in its ability to suppress unnecessary medication and therefore to contribute to reductions in medical expenses and burden on patients.

A preferred embodiment of the second aspect of the present invention also provides a method for determining a therapeutic effect on allergic conjunctivitis. The method for determination according to the present invention can be used to determine a therapeutic effect on allergic conjunctivitis using a lipid metabolite in a biological sample as an index.

In the method for determination according to the present invention, the step (C) of measuring the concentration of a lipid metabolite in a biological sample of a subject is carried out in a similar manner as in the method for detection of the present invention. The concentration of the lipid metabolite can be measured in a similar manner as in the method for detection of the present invention.

In the method for determination of the present invention, the step of determining a therapeutic effect on allergic conjunctivitis in the subject having undergone treatment based on the concentration of the lipid metabolite measured in the step (C) can further be carried out. In this step, when the concentration of the lipid metabolite in the biological sample of the subject having undergone treatment of allergic conjunctivitis is below the concentration of the lipid metabolite in the biological sample of the subject before the treatment or the concentration of the lipid metabolite in a biological sample of a subject suffering from allergic conjunctivitis (preferably, below with a significant difference), it is indicated that the treatment has a therapeutic effect. That is, the method for determination of the present invention may further comprise the step (D2) of determining that treatment of allergic conjunctivitis has a therapeutic effect, when the concentration of the lipid metabolite in the biological sample of the subject having undergone the treatment is below the concentration of the lipid metabolite in the biological sample of the subject before the treatment or the concentration of the lipid metabolite in a biological sample of a subject suffering from allergic conjunctivitis (preferably, below with a significant difference). As the concentration of the lipid metabolite in the biological sample of the subject before the treatment, there can be used a value obtained by measuring the concentration of the lipid metabolite in the biological sample of the subject before the treatment. As the concentration of the lipid metabolite in the biological sample of the healthy subject, there can be used a mean value calculated by collecting biological samples from a plurality of healthy subjects in advance and measuring the concentrations of the lipid metabolite in the biological samples. The subject who/which undergoes the treatment in the method for determination of the present invention is preferably a subject suffering from allergic conjunctivitis. In the present invention, the “subject suffering from allergic conjunctivitis” may be a subject demonstrated to have allergic conjunctivitis from results of other inspection methods, and can preferably be a subject diagnosed as developing allergic conjunctivitis by a doctor or veterinarian.

Examples of the treatment of allergic conjunctivitis on which a therapeutic effect can be determined by the method for determination of the present invention include drug therapy, immunotherapy and surgical treatment. The drug therapy includes treatment with therapeutic drugs for allergic conjunctivitis, and examples of such therapeutic drugs include antihistamines, leukotriene receptor antagonists and thromboxane A2 inhibitors. The immunotherapy includes administration of allergens, and examples of such allergens include pollen such as Japanese cedar pollen. Examples of administration forms include sublingual administration, subcutaneous administration and translymph node administration.

The method for determination according to the present invention can be used to determine the therapeutic effect in the subject having undergone treatment of allergic conjunctivitis, thereby verifying the effectiveness of the treatment of allergic conjunctivitis performed on the subject. Then, if no therapeutic effect is observed, the treatment can be immediately stopped and another treatment plan can be made. Therefore, the method for determination of the present invention is advantageous in its ability to suppress unnecessary medication and therefore to contribute to reductions in medical expenses and burden on patients.

According to a third aspect of the present invention, there is provided use of a lipid metabolite as an allergic disease marker. In the present invention, the term “allergic disease marker” refers to a substance of which the presence and amount serve as indicators of the presence or absence of development of the allergic disease or the severity of its symptom. In other words, according to the present invention, it is possible to use the lipid metabolite as a disease identification marker for an allergic disease, to use the lipid metabolite to differentiate the allergic disease from other similar diseases, and to use the lipid metabolite to evaluate the severity of the allergic disease.

A preferred embodiment of the third aspect of the present invention provides use of a lipid metabolite as an allergic rhinitis marker. In the present invention, the “allergic rhinitis marker” refers to a substance of which the presence and amount serve as indicators of the presence or absence of development of the allergic rhinitis or the severity of its symptom. In other words, according to the present invention, it is possible to use the lipid metabolite as a disease identification marker for allergic rhinitis, to use the lipid metabolite to differentiate allergic rhinitis from other similar diseases (for example, upper respiratory inflammation and asthma), and to use the lipid metabolite to evaluate the severity of allergic rhinitis.

The third aspect of the present invention also provides use of a lipid metabolite as an activation marker for mast cells and/or eosinophils in the nasal cavities. In the present invention, the term “activation marker for mast cells and/or eosinophils in the nasal cavities” refers to a substance of which the presence and amount serve as indicators of the presence or absence of activation of mast cells in the nasal cavities or the degree of activation thereof. In other words, according to the present invention, it is possible to use the lipid metabolite as an activation marker for mast cells and/or eosinophils in the nasal cavities, and to use the lipid metabolite in the inspection of the degree of allergic reaction within the nasal cavities in addition to the presence or absence of activation of mast cells and/or eosinophils in the nasal cavities or the degree of activation thereof. The use of the present invention can be carried out in accordance with the descriptions concerning the method for detection and method for determination according to the present invention.

A preferred embodiment of the third aspect of the present invention also provides use of a lipid metabolite as an allergic conjunctivitis marker. In the present invention, the “allergic conjunctivitis marker” refers to a substance of which the presence and amount serve as indicators of the presence or absence of development of allergic conjunctivitis or the severity of its symptom. In other words, according to the present invention, it is possible to use the lipid metabolite as a disease identification marker for allergic conjunctivitis, to use the lipid metabolite to differentiate allergic conjunctivitis from other similar diseases (for example, infectious conjunctivitis), and to use the lipid metabolite to evaluate the severity of allergic conjunctivitis.

The third aspect of the present invention also provides use of a lipid metabolite as an activation marker for mast cells and/or eosinophils in the eyes. In the present invention, the term “activation marker for mast cells and/or eosinophils in the eyes” refers to a substance of which the presence and amount serve as indicators of the presence or absence of activation of mast cells in the eyes or the degree of activation thereof. In other words, according to the present invention, it is possible to use the lipid metabolite as an activation marker for mast cells and/or eosinophils in the eyes, and to use the lipid metabolite in the inspection of the degree of allergic reaction within the eyes in addition to the presence or absence of activation of mast cells and/or eosinophils in the eyes or the degree of activation thereof. The use of the present invention can be carried out in accordance with the descriptions concerning the method for detection and method for determination according to the present invention.

The method for determination of the present invention can be used to determine the effectiveness of a therapeutic drug for an allergic disease, and thus can be used also to screen for a therapeutic drug for an allergic disease. For example, the method for determination of the present invention can be used to determine the effectiveness of a therapeutic drug for allergic rhinitis, and thus can be used also to screen for a therapeutic drug for allergic rhinitis. That is, according to a fourth aspect of the present invention, there is provided a method for screening for a therapeutic drug for allergic rhinitis, comprising the step (E1) of administering a candidate drug for a therapeutic drug for allergic rhinitis to a subject and the step (F1) of measuring the concentration of a lipid metabolite in a biological sample of the subject. In the method for screening of the present invention, the step of determining whether the candidate drug has a therapeutic effect based on the concentration of the lipid metabolite measured in the step (F1) can further be carried out. In this step, when the concentration of the lipid metabolite in the biological sample of the subject after the administration of the candidate drug is below the concentration of the lipid metabolite in the biological sample of the subject before the administration of the candidate drug or the concentration of the lipid metabolite in a biological sample of a subject suffering from allergic rhinitis (preferably, below with a significant difference), it is indicated that the candidate drug has a therapeutic effect. That is, the method for screening of the present invention may further comprise the step (G1) of determining that the candidate drug has a therapeutic effect, when the concentration of the lipid metabolite in the biological sample of the subject after the administration of the candidate drug is below the concentration of the lipid metabolite in the biological sample of the subject before the administration of the candidate drug or the concentration of the lipid metabolite in a biological sample of a subject suffering from allergic rhinitis (preferably, below with a significant difference). By virtue of this step, a candidate drug having a therapeutic effect can be selected. The method for screening of the present invention can be carried out in accordance with the descriptions concerning the method for detection and method for determination according to the present invention. The subject to which the candidate drug is administered in the method for screening of the present invention is preferably a subject suffering from allergic rhinitis. When the method for screening of the present invention is carried out, mammals other than humans can be used as the subject.

Also, the method for determination of the present invention can be used to determine the effectiveness of a therapeutic drug for allergic conjunctivitis, and thus can be used also to screen for a therapeutic drug for allergic conjunctivitis. That is, according to the fourth aspect of the present invention, there is also provided a method for screening for a therapeutic drug for allergic conjunctivitis, comprising the step (E2) of administering a candidate drug for a therapeutic drug for allergic conjunctivitis to a subject and the step (F2) of measuring the concentration of a lipid metabolite in a biological sample of the subject. In the method for screening of the present invention, the step of determining whether the candidate drug has a therapeutic effect based on the concentration of the lipid metabolite measured in the step (F2) can further be carried out. In this step, when the concentration of the lipid metabolite in the biological sample of the subject after the administration of the candidate drug is below the concentration of the lipid metabolite in the biological sample of the subject before the administration of the candidate drug or the concentration of the lipid metabolite in a biological sample of a subject suffering from allergic conjunctivitis (preferably, below with a significant difference), it is indicated that the candidate drug has a therapeutic effect. That is, the method for screening of the present invention may further comprise the step (G2) of determining that the candidate drug has a therapeutic effect, when the concentration of the lipid metabolite in the biological sample of the subject after the administration of the candidate drug is below the concentration of the lipid metabolite in the biological sample of the subject before the administration of the candidate drug or the concentration of the lipid metabolite in a biological sample of a subject suffering from allergic conjunctivitis (preferably, below with a significant difference). By virtue of this step, a candidate drug having a therapeutic effect can be selected. The method for screening of the present invention can be carried out in accordance with the descriptions concerning the method for detection and method for determination according to the present invention. The subject to which the candidate drug is administered in the method for screening of the present invention is preferably a subject suffering from allergic conjunctivitis. When the method for screening of the present invention is carried out, mammals other than humans can be used as the subject.

According to a fifth aspect of the present invention, there is provided a method for identifying, in a lipid metabolite in a biological sample, an allergic disease marker or an activation marker for mast cells and/or eosinophils in the nasal cavities or eyes, comprising the step (H) of measuring the concentration of a lipid metabolite in a biological sample of a subject suffering from an allergic disease and the concentration of the lipid metabolite in a biological sample of a healthy subject; and the step (I) of comparing the measured two concentrations.

A preferred embodiment of the fifth aspect of the present invention provides a method for identifying, in a lipid metabolite in a biological sample, an allergic rhinitis marker or an activation marker for mast cells and/or eosinophils in the nasal cavities, comprising the step (H1) of measuring the concentration of a lipid metabolite in a biological sample of a subject suffering from allergic rhinitis and the concentration of the lipid metabolite in a biological sample of a healthy subject; and the step (I1) of comparing the measured two concentrations. In the method for identification of the present invention, the step of determining that the lipid metabolite is an allergic rhinitis marker or an activation marker for mast cells and/or eosinophils in the nasal cavities based on the results of comparison between the concentrations performed in the step (I1) can further be carried out. In this step, when the concentration of the lipid metabolite in the biological sample of the subject suffering from allergic rhinitis is above the concentration of the lipid metabolite in the biological sample of the healthy subject (preferably, above with a significant difference), it is indicated that the lipid metabolite is an allergic rhinitis marker or an activation marker for mast cells and/or eosinophils in the nasal cavities. That is, the method for identification of the present invention may further comprise the step (J1) of determining that the lipid metabolite is an allergic rhinitis marker or an activation marker for mast cells and/or eosinophils in the nasal cavities, when the concentration of the lipid metabolite in the biological sample of the subject suffering from allergic rhinitis is above the concentration of the lipid metabolite in the biological sample of the healthy subject (preferably, above with a significant difference). The method for identification of the present invention can be carried out in accordance with the descriptions concerning the method for detection and method for determination according to the present invention.

A preferred embodiment of the fifth aspect of the present invention also provides a method for identifying, in a lipid metabolite in a biological sample, an allergic conjunctivitis marker or an activation marker for mast cells and/or eosinophils in the eyes, comprising the step (H2) of measuring the concentration of a lipid metabolite in a biological sample of a subject suffering from allergic conjunctivitis and the concentration of the lipid metabolite in a biological sample of a healthy subject, and the step (12) of comparing the measured two concentrations. In the method for identification of the present invention, the step of determining that the lipid metabolite is an allergic conjunctivitis marker or an activation marker for mast cells and/or eosinophils in the eyes based on the results of comparison between the concentrations performed in the step (I2) can further be carried out. In this step, when the concentration of the lipid metabolite in the biological sample of the subject suffering from allergic conjunctivitis is above the concentration of the lipid metabolite in the biological sample of the healthy subject (preferably, above with a significant difference), it is indicated that the lipid metabolite is an allergic conjunctivitis marker or an activation marker for mast cells and/or eosinophils in the eyes. That is, the method for identification of the present invention may further comprise the step (J2) of determining that the lipid metabolite is an allergic conjunctivitis marker or an activation marker for mast cells and/or eosinophils in the eyes, when the concentration of the lipid metabolite in the biological sample of the subject suffering from allergic conjunctivitis is above the concentration of the lipid metabolite in the biological sample of the healthy subject (preferably, above with a significant difference). The method for identification of the present invention can be carried out in accordance with the descriptions concerning the method for detection and method for determination according to the present invention.

Treatment of an allergic disease can be performed on the subject in which the allergic disease has been detected by the method for detection of the present invention. According to a sixth aspect of the present invention, there is therefore provided a method for treating an allergic disease, comprising the step (A) of measuring the concentration of a lipid metabolite in a biological sample of a subject; the step (B) of determining that the subject suffers from the allergic disease when the concentration of the lipid metabolite in the biological sample of the subject is above the concentration of the lipid metabolite in a biological sample of a healthy subject; and the step (K) of performing treatment of the allergic disease on the subject determined to suffer from the allergic disease.

A preferred embodiment of the sixth aspect of the present invention provides a method for treating allergic rhinitis, comprising the step (A) of measuring the concentration of a lipid metabolite in a biological sample of a subject; the step (B1) of determining that the subject suffers from allergic rhinitis when the concentration of the lipid metabolite in the biological sample of the subject is above the concentration of the lipid metabolite in a biological sample of a healthy subject; and the step (K1) of performing treatment of allergic rhinitis on the subject determined to suffer from allergic rhinitis. The steps of detecting allergic rhinitis (i.e., steps (A) and (B1)) in the method for treatment of the present invention can be carried out in accordance with the descriptions concerning the method for detection according to the present invention. The treatment of allergic rhinitis can be performed according to the descriptions concerning the method for determination of the present invention.

A preferred embodiment of the sixth aspect of the present invention also provides a method for treating allergic conjunctivitis, comprising the step (A) of measuring the concentration of a lipid metabolite in a biological sample of a subject; the step (B2) of determining that the subject suffers from allergic conjunctivitis when the concentration of the lipid metabolite in the biological sample of the subject is above the concentration of the lipid metabolite in a biological sample of a healthy subject; and the step (K2) of performing treatment of allergic conjunctivitis on the subject determined to suffer from allergic conjunctivitis. The steps of detecting allergic conjunctivitis (i.e., steps (A) and (B2)) in the method for treatment of the present invention can be carried out in accordance with the descriptions concerning the method for detection according to the present invention. The treatment of allergic conjunctivitis can be performed according to the descriptions concerning the method for determination of the present invention.

According to a seventh aspect of the present invention, there is provided a kit for detecting an allergic disease (for example, allergic rhinitis or allergic conjunctivitis) or an activation state of mast cells and/or eosinophils in the nasal cavities or eyes, comprising a means for quantifying a lipid metabolite in a biological sample of a subject. The kit of the present invention is typically a kit for detecting an allergic disease (for example, allergic rhinitis or allergic conjunctivitis) and an activation state of mast cells and/or eosinophils in the nasal cavities or eyes using the method for detection of the present invention. Examples of the means for quantifying a lipid metabolite include a substance that specifically binds to the lipid metabolite, and the means for quantification is typically an antibody against the lipid metabolite. As the means for quantifying a lipid metabolite, a mass spectrometer for use in the mass spectrometry as described above is also indicated.

When the means for quantifying a lipid metabolite is an antibody in the kit of the present invention, the kit of the present invention comprises a reagent (and a device in some cases) necessary for measuring the concentration of the lipid metabolite by immunoassay utilizing the antibody.

Examples of the kit of the present invention include a kit that measures the concentration of the lipid metabolite by a sandwich method. The kit may comprise a microtiter plate, an anti-lipid metabolite antibody for capture, an anti-lipid metabolite antibody labeled with alkaline phosphatase or peroxidase, and an alkaline phosphatase substrate or peroxidase substrate.

The kit described above can be used, for example, in the following manner. First, the antibody for capture is fixed on the microtiter plate, and a biological sample of a subject is appropriately diluted and added thereto, and then incubated. The sample is removed and washed. Subsequently, the labeled anti-lipid metabolite antibody is added. After incubation and washing, the substrate is added for color development. The color development can be measured using a microtiter plate reader or the like to determine the concentration of the lipid metabolite.

Examples of the kit of the present invention also include a kit that measures the concentration of the lipid metabolite by a sandwich method using a secondary antibody. The kit may comprise a microtiter plate, an anti-lipid metabolite antibody for capture, an anti-lipid metabolite antibody as a primary antibody, an antibody against an anti-lipid metabolite antibody labeled with alkaline phosphatase or peroxidase as a secondary antibody, and an alkaline phosphatase substrate or peroxidase substrate.

The kit described above can be used, for example, in the following manner. First, the antibody for capture is fixed on the microtiter plate, and a biological sample of a subject is appropriately diluted and added thereto, and then incubated. The sample is removed and washed. Subsequently, the primary antibody is added, and incubation and washing are performed. The enzyme-labeled secondary antibody is further added. After incubation, the substrate is added for color development. The color development can be measured using a microtiter plate reader or the like to determine the concentration of the lipid metabolite.

In the kit of the present invention, the labeled antibody is not limited to the enzyme-labeled antibody, and may be an antibody labeled with a radioactive substance (such as ²⁵I, ¹³¹I, ³⁵S or ³H), a fluorescent substance (such as fluorescein isothiocyanate, rhodamine, dansyl chloride, phycoerythrin, tetramethylrhodamine isothiocyanate, or near-infrared fluorescent material), a luminescent substance (such as luciferase, luciferin or equolin), a nanoparticle (such as colloidal gold or quantum dot), or the like. It is also possible to use a biotinylated antibody as the labeled antibody and to add labeled avidin or streptavidin to the kit.

Further examples of the kit of the present invention include a kit that measures the concentration of a lipid metabolite by an immunochromatography method. The kit can have a structure in which an antibody storage part in which a first anti-lipid metabolite antibody labeled with colloidal gold or the like is stored and a determination part in which a second anti-lipid metabolite antibody (preferably, an antibody that recognizes another epitope of the lipid metabolite) is fixed in a line on a cellulose membrane or the like are connected by a narrow groove.

The kit described above can be used, for example, in the following manner. First, when a biological sample is added to the antibody storage part or a biological sample receiving part adjacent to the antibody storage part, the labeled antibody and the lipid metabolite are bound in the antibody storage part to form a lipid metabolite-labeled antibody complex. The complex moves to the determination part through the groove due to a capillary phenomenon. Subsequently, when the complex is captured by the fixed second anti-lipid metabolite antibody, a red line appears in the determination part due to the plasmon effect of colloidal gold, so that the presence of the lipid metabolite can be detected. The kit, in this case, can be provided in the form of a stick, such as a stick for inspection, and urine can be used as the biological sample. The stick for inspection may further be equipped with an absorbent paper for absorbing a urine specimen, a desiccant and the like.

When the means for quantifying a lipid metabolite is a mass spectrometer in the kit of the present invention, the kit of the present invention comprises an internal standard device, in some cases, in addition to the mass spectrometer. An internal standard can be used to correct the extraction efficiency and ionization efficiency for each analysis at the time of measurement with a mass spectrometer. The internal standard used in mass spectrometry includes deuterated lipid metabolites.

The kit of the present invention can be implemented in accordance with the descriptions concerning the method for detection and method for determination according to the present invention, in addition to the above description.

The present invention provides the following inventions.

[101] A method for detecting allergic rhinitis or an activation state of mast cells and/or eosinophils in the nasal cavities, comprising the step of measuring the concentration of a lipid metabolite in a biological sample of a subject.

[102] The method for detection according to [101], wherein, when the concentration of the lipid metabolite in the biological sample of the subject is above the concentration of the lipid metabolite in a biological sample of a healthy subject, it is indicated that the subject suffers from allergic rhinitis or that mast cells and/or eosinophils are activated in the nasal cavities of the subject.

[103] A method for determining a therapeutic effect on allergic rhinitis, comprising the step of measuring the concentration of a lipid metabolite in a biological sample of a subject.

[104] The method for determination according to [103], wherein, when the concentration of the lipid metabolite in the biological sample of the subject is below the concentration of the lipid metabolite in the biological sample of the subject before treatment or the concentration of the lipid metabolite in a biological sample of a subject suffering from allergic rhinitis, it is indicated that the treatment has a therapeutic effect.

[105] The method for determination according to any one of [103] to [104], wherein the treatment of allergic rhinitis is drug therapy, immunotherapy or surgical treatment.

[106] The method for detection according to [101] or [102], or the method for determination according to any one of [103] to [105], wherein the biological sample is a body fluid noninvasively collected from the subject.

[107] The method for detection according to any one of [101], [102] and [106], or the method for determination according to any one of [103] or [106], wherein the allergic rhinitis is seasonal allergic rhinitis or perennial allergic rhinitis.

[108] The method for detection according to any one of [101], [102], [106] and [107], or the method for determination according to any one of [103] to [107], wherein the lipid metabolite is one or more lipid metabolite(s) selected from the group consisting of an arachidonic acid metabolite, an eicosapentaenoic acid metabolite, a docosahexaenoic acid metabolite, a linolenic acid metabolite and an eicosadienoic acid metabolite.

[109] The method for detection according to any one of [101], [102] and [106] to [108], or the method for determination according to any one of [103] to [108], wherein the lipid metabolite is one or more lipid metabolite(s) selected from the group consisting of PGD₂, PGE₂, 12-HETE, 15-HETE, 12-HEPE, 15-HEPE, 14-HDOHE, 8-HDoHE, 13-HODE, 8-isoPGE₂, 16-HDoHE, 15-HEDE, 15-OxoEDE, 20-HETE and 14,15-EET.

[110] The method for detection according to any one of [101], [102] and [106] to [109], or the method for determination according to any one of [103] to [109], wherein the concentration of the lipid metabolite is measured by mass spectrometry.

[111] Use of a lipid metabolite as an allergic rhinitis marker or an activation marker for mast cells and/or eosinophils in the nasal cavities.

[112] The use according to [111], wherein the allergic rhinitis is seasonal allergic rhinitis or perennial allergic rhinitis.

[113] The use according to [111] or [112], wherein the lipid metabolite is one or more lipid metabolite(s) selected from the group consisting of an arachidonic acid metabolite, an eicosapentaenoic acid metabolite, a docosahexaenoic acid metabolite, a linolenic acid metabolite and an eicosadienoic acid metabolite.

[114] The use according to any one of [111] to [113], wherein the lipid metabolite is one or more lipid metabolite(s) selected from the group consisting of PGD₂, PGE₂, 12-HETE, 15-HETE, 12-HEPE, 15-HEPE, 14-HDOHE, 8-HDOHE, 13-HODE, 8-isoPGE₂, 16-HDoHE, 15-HEDE, 15-OxoEDE, 20-HETE and 14,15-EET.

[115] A method for screening for a therapeutic drug for allergic rhinitis, comprising the steps of administering a candidate drug for a therapeutic drug for allergic rhinitis to a subject; and measuring the concentration of a lipid metabolite in a biological sample of the subject.

[116] The method for screening according to [115], wherein, when the concentration of the lipid metabolite in the biological sample of the subject after the administration of the candidate drug is below the concentration of the lipid metabolite in the biological sample of the subject before the administration of the candidate drug or the concentration of the lipid metabolite in a biological sample of a subject suffering from allergic rhinitis, it is indicated that the therapeutic drug has a therapeutic effect.

[117] A method for identifying, in a lipid metabolite in a biological sample, an allergic rhinitis marker or an activation marker for mast cells and/or eosinophils in the nasal cavities, comprising the steps of measuring the concentration of a lipid metabolite in a biological sample of a subject suffering from allergic rhinitis and the concentration of the lipid metabolite in a biological sample of a healthy subject; and comparing the measured two concentrations.

[118] The method for identification according to [117], wherein, when the concentration of the lipid metabolite in the biological sample of the subject suffering from allergic rhinitis is above the concentration of the lipid metabolite in the biological sample of the healthy subject, it is indicated that the lipid metabolite serves as an allergic rhinitis marker or an activation marker for mast cells and/or eosinophds in the nasal cavities.

### EXAMPLES

Hereinafter, the present invention will be described in more detail by way of the following examples, but is not limited thereto.

### Example 1: Preparation of Disease Model Mice

### (1) Preparation of Allergic Rhinitis Model Mice

To 23 BALB/C mice (female, 5 weeks old) (CLEA Japan, Inc., the same applies hereinafter), 50 μg of ovalbumin (manufactured by Sigma, sometimes abbreviated as “OVA” herein) dissolved and diluted with physiological saline (manufactured by Otsuka Pharmaceutical Co., Ltd.) and 1 mg of aluminum potassium sulfate (manufactured by Sigma-Aldrich Co. LLC.) were intraperitoneally administered simultaneously, and, after two weeks, 50 μg of OVA was intraperitoneally administered again for sensitization. Two weeks after the second sensitization, OVA (400 μg/10 μL) was transnasally administered once daily for 5 days (5 times, in total) or 10 days (10 times, in total) for stimulation, thereby preparing allergic rhinitis (sometimes abbreviated as “AR” herein) model mice. A group to which OVA was administered 5 times was defined as “5-time OVA-administered group” (11 mice), and a group to which OVA was administered 10 times was defined as “10-time OVA-administered group” (12 mice).

### (2) Preparation of Upper Respiratory Inflammation Model Mice

Lipopolysaccharide (manufactured by Sigma, sometimes abbreviated as “LPS” herein) (15 μg/10 μL) was transnasally administered once to 9 BALB/C mice (female, 5 weeks old), thereby preparing upper respiratory inflammation model mice.

### (3) Control Group

As a control group, the physiological saline (10 μL) used in the above item (1) was transnasally administered to 10 BALB/C mice (female, 5 weeks old) once daily for 1 day (once, in total), 5 days (5 times, in total), or 10 days (10 times, in total) (solvent-administered group). A group to which the solvent was administered once was defined as “single-time solvent-administered group,” a group to which the solvent was administered 5 times was defined as “5-time solvent-administered group, and a group to which the solvent was administered 10 times was defined as “10-time solvent-administered group.”

### Example 2: Evaluation of Symptoms of Allergic Rhinitis and Upper Respiratory, Inflammation

### (1) Measurement of Antigen-Specific IgE Level in Serum

Antigen sensitization is known to increase the antigen-specific IgE value in serum. To confirm the ovalbumin sensitization of the allergic rhinitis model mice prepared in Example 1 (1), the ovalbumin-specific IgE level in serum was measured. Specifically, 50 μL of blood was collected from the orbits of OVA-sensitized mice (a group not stimulated with OVA after two sensitizations with OVA) (5 mice) and unsensitized mice (BALB/C mice (female, 5 weeks old), 5 mice, sometimes referred to as “unsensitized group” herein) as a control group. The collected blood was left at an ordinary temperature for 1 hour. The blood was then centrifuged at 9500 rpm and 4° C. for 10 minutes to separate serum from blood clot. Serum was collected and then stored at −80° C. The freeze-preserved serum was diluted 25 times, and then the IgE level was measured using Anti-Ovalbumin IgE (mouse) ELISA Kit (manufactured by Cayman Chemical Company) according to the instructions attached to the kit.

### (2) Evaluation of Sneezing Symptom

For the AR model mice (22 mice) and solvent-administered group (10 mice) prepared in Example 1, the numbers of sneezes for a period of 10 minutes from 1 minute to 11 minutes after each of the first to tenth administrations of OVA or the solvent were counted. For the upper respiratory inflammation model mice (9 mice) and single-time solvent-administered group (6 mice) prepared in Example 1, the numbers of sneezes were counted hourly after the administration of LPS or the solvent (until 10 hours later).

### (3) Measurement of Nasal Cavity Volume

The nasal cavity volume of the AR model mice prepared in Example 1 was measured by computed tomography before the administration of OVA (6 mice), 24 hours after the fifth administration of OVA (6 mice), and 24 hours after the tenth administration of OVA (6 mice). As a control, the nasal cavity volume of the solvent-administered group prepared in Example 1 was measured in the same manner as above before the administration of the solvent (6 mice), 24 hours after the fifth administration of the solvent (6 mice), and 24 hours after the tenth administration of the solvent (6 mice). Specifically, anesthetized mice were fixed on a 48-mm Φ visual field fixture, and then imaged using a micro CT (LCT-200, manufactured by Hitachi Aloka Medical Ltd.). The imaging conditions were as follows: pixel size: 48 μm; slice thickness/slice interval: 96 μm; number of slices: 35; rotation speed: standard; rotation angle: 360°; X-tube voltage: low; and number of projection directions: 1592. From the captured image, regions having a CT value of −900 to −300 HU (Hounsfield unit) were decided as the nasal cavities, and their volume was measured.

### (4) Statistical Processing

In Examples 2 to 5, measured values were expressed as mean value±standard deviation. Also, as a significance test, one-way analysis of variance (ANOVA) was used, followed by a Tukey's test. As a significance test between two-factor groups, two-way ANOVA was used, followed by a Bonferroni post-test. When the risk rate (P) was less than 5%, it was determined that there was a significant difference.

### (5) Results

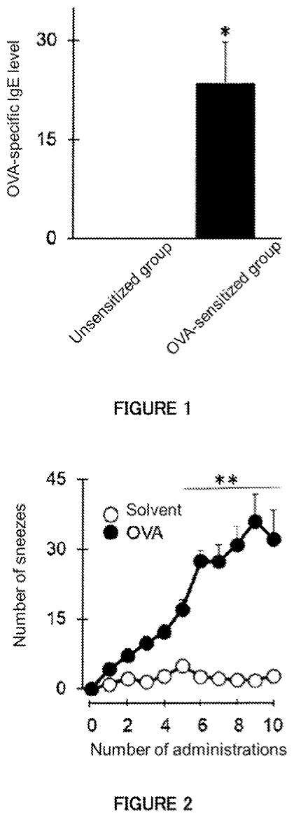

The results of the evaluations in the above items (1) to (3), which were performed on the model mice prepared in Example 1, were as shown in FIGS. 1 to 4.

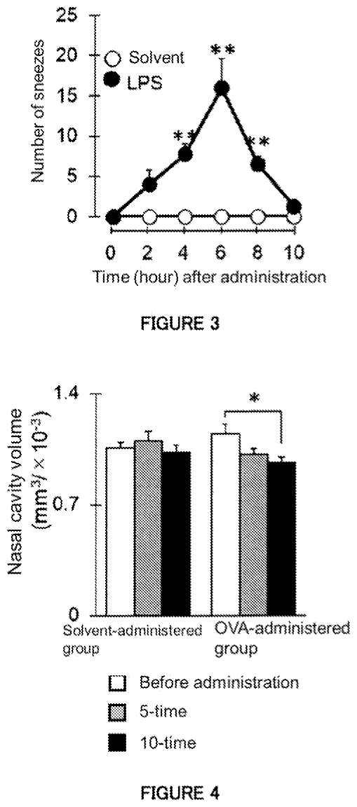

From the results shown in FIG. 1, the OVA-specific IgE was not detected in the unsensitized group, whereas 23±6.29 ng/mL of serum ovalbumin-specific IgE was detected in the OVA-sensitized mice. From this fact, it was confirmed that the allergic rhinitis model mice prepared in Example 1 (1) were sensitized with ovalbumin. In addition, from the results shown in FIG. 2, the number of administrations of the solvent did not affect the frequency of sneezing in the solvent-administered group, whereas the frequency of sneezing was observed to increase from the first administration of OVA in the allergic rhinitis model mice. In the fifth administration of OVA, the number of sneezes significantly increased as compared with that of the solvent-administered group, and a large number of sneezes was maintained until the tenth administration of OVA. In addition, from the results shown in FIG. 3, the number of sneezes of the upper respiratory inflammation model mice increased 2 hours after the administration of LPS and reached the maximum value 6 hours after the administration of LPS, but decreased to the level equivalent to that of the solvent-administered group 10 hours after the administration of LPS. The maximum value of the number of sneezes of the upper respiratory inflammation model mice was similar to the number of sneezes of the allergic rhinitis model mice in the fifth administration of OVA (5-time OVA-administered group). Moreover, from the results shown in FIG. 4, the number of administrations of the solvent did not affect the nasal cavity volume, in the solvent-administered group. In the allergic rhinitis model mice, the nasal cavity volume decreased in the 5-time OVA-administered group, and significantly decreased in the 10-time OVA-administered group as compared with that before the administration of OVA. From this fact, the symptom of nasal congestion was confirmed in the 10-time OVA-administered group.

### Example 3: Histopathological Evaluation of Allergic Rhinitis and Upper Respiratory, Inflammation

### (1) Procedures

Histopathological analysis was performed using the AR model mice (5-time OVA-administered group and 10-time OVA-administered group), upper respiratory inflammation model mice (LPS-administered group), and control groups (single-time solvent-administered group, 5-lime solvent-administered group, and 10-time solvent-administered group) (6 mice in each of the groups) which were prepared in Example 1. Specifically, the maxilla was excised from the mice and immediately immersed in 4% paraformaldehyde at 4° C. for 5 days. Then, it was immersed in EDTA every day and decalcified at 4° C. for 10 days. Next, the bone covering the orbit was excised with a razor, dehydrated and cleared, and then embedded in paraffin to prepare continuous section specimens having a width of 4 μm. Hematoxylin-eosin staining (HE staining), chloroacetate-esterase staining (CAE staining), toluidine blue staining (TB staining) or May-Grünwald Giemsa staining (MG staining) was performed according to a conventional method for encapsulation. Specific staining procedures are as will be described below. The stained nasal cavity tissue was observed under an optical microscope (ACT-10, manufactured by Nikon Corporation), and imaged using a digital microscope camera (OPTIPHOT-2, manufactured by Nikon Corporation). The number of immunocytes (connective tissue type mast cells, mucosal type mast cells, eosinophils and neutrophils) in the nasal turbinates (sites where inflammatory cells infiltrate when rhinitis occurs and where many capillaries gather) was counted within 16 to 24 randomly selected areas (magnification: ×400) per section.

### (2) Chloroacetate-Esterase (CAE) Staining

CAE staining is a staining method in which mast cell-specific esterase is stained red using naphthol AS-D chloroacetate as a substrate. The continuous section specimens prepared in the above item (1) were deparaffinized and used for staining. A 4% sodium nitrite solution and a new fuchsin solution were mixed at a ratio of 1:1, and then this solution and a naphthol solution were mixed at a ratio of 1:9. This solution and phosphate buffer (0.2M NaH₂PO₄, 0.2M Na₂HPO₄, pH 7.6) were mixed at a ratio of 1:20, and the sections were immersed for 10 minutes. The sections were washed with distilled water and then counterstained with a hematoxylin solution. The total number of the mast cells (connective tissue type mast cells and mucosal type mast cells) present in the connective tissue and mucosal tissue within 1 mm² was counted to calculate the mean value and standard deviation.

### (3) Toluidine Blue (TB) Staining

TB staining stains acidic mucous polysaccharides. Acidic mucous polysaccharides are present in the connective tissue type mast cells and absent in the mucosal type mast cells. The continuous section specimens prepared in the above item (1) were deparaffinized and then immersed in a 0.05% toluidine blue solution (pH: 4.1) for 10 minutes. Then, they were decolorized with a 90% alcohol solution and then encapsulated. CAE staining stains both the connective tissue type mast cells and the mucosal type mast cells, whereas TB staining stains only the connective tissue type mast cells. CAE staining and TB staining were performed on different sections, and cells positive for CAE staining and positive for TB staining were decided as connective tissue type mast cells. Also, cells positive for CAE staining and negative for TB staining were decided as mucosal type mast cells. The total number of the connective tissue mast cells or mucosal type mast cells present in the connective tissue and mucosal tissue within 1 mm² was counted to calculate the mean value and standard deviation.

### (4) May-Grünwald Giemsa (MG) Staining

May-Grünwald Giemsa staining stains eosinophil-specific eosinophilic granules red. The continuous section specimens prepared in the above item (1) were deparaffinized and then immersed in a May-Grünwald staining solution for 6 minutes. Then, the sections were washed with 1/15 M-phosphate buffer (×10) (0.2M NaH₂PO₄, 0.2M Na₂HPO₄, pH 6.4 to 6.8). Thereafter, the sections were immersed for 3 minutes in a solution obtained by mixing a Giemsa staining solution and distilled water at a ratio of 1:20. HE staining and MG staining were performed on different sections, and cells confirmed to have a multilobed nucleus by HE staining and positive for MG staining were decided as eosinophils. Also, cells have a multilobed nucleus by HE staining and negative for MG staining were decided as neutrophils. The total number of the eosinophils or neutrophils present in the connective tissue and mucosal tissue within 1 mm² was counted to calculate the mean value and standard deviation,

### (5) Results

The results of the histopathological evaluations in the above items (2) to (4), which were performed on the model mice prepared in Example 1, were as shown in FIGS. 6 to 9.

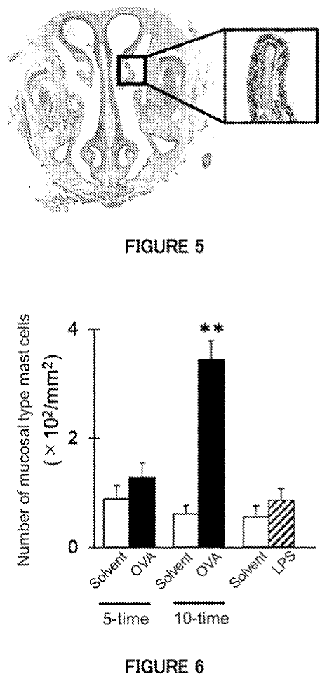

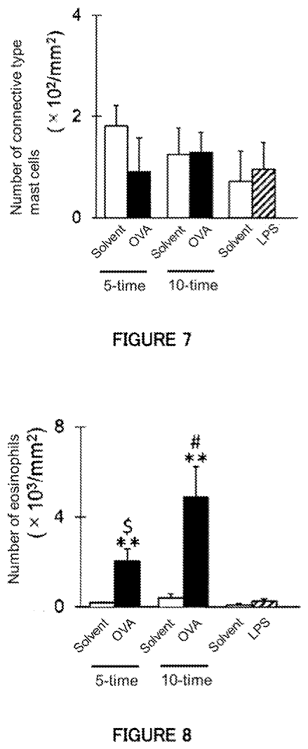

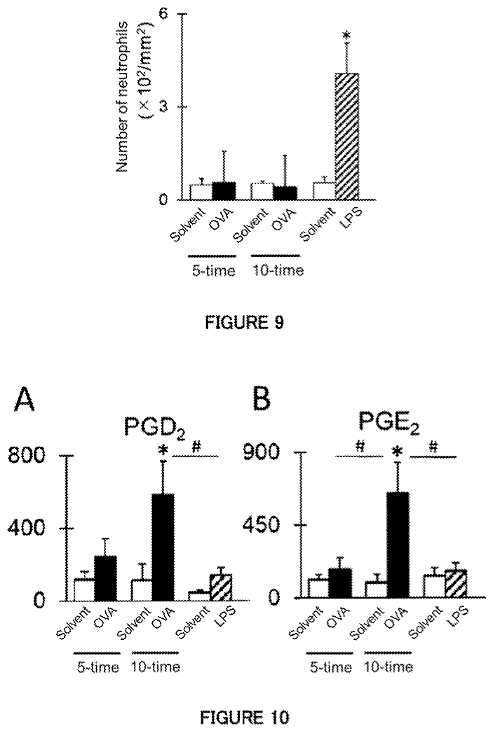

As shown in FIG. 6, the 5-time OVA-administered group, the solvent-administered groups (number of administrations: 1, 5 and 10 times, the same applies hereinafter) and the LPS-administered group showed no significant difference in number of mucosal type mast cells infiltrated into the nasal turbinates (FIG. 5). On the other hand, the 10-time OVA-administered group showed a significant increase in number of mucosal type mast cells as compared with the 10-time solvent-administered group. In addition, as shown in FIG. 7, the OVA-administered groups (5 and 10 times), the solvent-administered groups (1, 5 and 10 times) and the LPS-administered group showed no significant difference in number of connective tissue type mast cells infiltrated into the nasal turbinates. Additionally, as shown in FIG. 8, the 5-time OVA-administered group and the 10-time OVA-administered group showed a significant increase in number of eosinophils infiltrated into the nasal turbinates as compared with the 10-time solvent-administered group. Further, the number of infiltrated eosinophils significantly increased in the 10-time OVA-administered group as compared with that in the 5-time OVA-administered group. Furthermore, the number of the infiltrated eosinophils significantly increased in the 5-time OVA-administered group as compared with that in the LPS-administered group. In addition, as shown in FIG. 9, no significant difference in the number of neutrophils infiltrated into the nasal turbinate was observed in the OVA-administered groups (5 and 10 times) and the solvent-administered groups (1, 5 and 10 times). In contrast, the LPS-administered group showed a significant increase in number of neutrophils as compared with the single-time solvent-administered group. As a result of comprehensive decision on the degree of intranasal (nasal turbinates) infiltration of the immunocytes, the pathological characteristics of the nasal cavity tissue, and the allergic symptoms (sneezing and nasal congestion) presented in Example 2, the 5-time OVA-administered group was defined as suffering from mild allergic rhinitis, and the 10-time OVA-administered group was defined as suffering from severe allergic rhinitis. It is known that the infiltration of mast cells and eosinophils into the tissue plays an important role in the onset and progress of allergic rhinitis, that highly mature connective type mast cells are resident in the nasal cavities, and that immature mucosal type mast cells are infiltrated into the nasal turbinates by inflammatory stimulation (Terada, N. et al., Journal of Allergy and Clinical Immunology 94,629-642, 1994).

### Example 4: Analysis of Lipid Mediator in Nasal Lavage Fluids of Allergic Rhinitis and Upper Respiratory Inflammation Model Mice

### (1) Measurement of Lipid Mediator

### A. Preparation of Nasal Lavage Fluid

The amounts of lipid mediators produced at the downstream of fatty acid metabolizing enzymes (COX, LOX and CYP) and under OX were inspected using the allergic rhinitis model mice (5-time OVA-administered group: 11 mice; and 10-time OVA-administered group: 12 mice), upper respiratory inflammation model mice (LPS-administered group: 9 mice), and solvent-administered groups (single-time solvent-administered group, 5-time solvent-administered group, and 10-time solvent-administered group: 10 mice, each) prepared in Example 1. The mice were euthanized 20 minutes after the administration of OVA, LPS or the solvent in each of the groups. A nasal lavage fluid was prepared by excising the mandible, flowing 550 μL of cooled physiological saline from the bronchi to the tip of the nose, collecting it, and freezing it at −80° C.

B. Lipid Extraction from Nasal Lavage Fluid

The lavage fluid prepared in the above item A was centrifuged at 1000 rpm and 4° C. for 10 minutes. A sample solution was prepared by adding 50 μL of 0.05% formic acid water, 50 μL of the internal standard solution indicated in Table 2 and 550 μL of distilled water to 400 μL of the supernatant. The sample solution was loaded onto a solid-phase extraction cartridge (HLB lee Vac Cartridge, Oasis, manufactured by Waters Corporation) equilibrated with methanol and distilled water in advance. After the cartridge was washed with 1 mL of MilliQ water and 1 mL of hexane, the lipid attached to the cartridge was eluted with 1 mL of methanol. Then, the eluate was dried and hardened under reduced pressure at room temperature for 4 hours to be formed into pellets. Then, the pellets were redissolved in 80% methanol to prepare a lipid sample solution.

### C. Measurement of Lipid Concentration by Liquid Chromatography-Mass Spectrometry

The sample solution prepared in the above item B was analyzed by a mass analyzer. The conditions used in liquid chromatography-mass spectrometry are as follows.

### <LC Conditions>

Analytical column: Phenomenex, Kinetex C8 (2.1 mm I.D.×150 mm, 2.6 μm, manufactured by KINETEX)
Mobile phase A: 0.05% formic acid
Mobile phase B: 0.05% acetonitrile formate
Flow rate: 0.4 mL/min
Injection volume: 5 μL
Column temperature: 45° C.
Gradient program: as indicated in Table 1

TABLE 1
Gradient program

| Step | Time (min) | Mobile phase A (%) | Mobile phase B (%) |
|---|---|---|---|
| 0 | 0 | 90 | 10 |
| 1 | 5 | 75 | 25 |
| 2 | 10 | 65 | 35 |
| 3 | 20 | 25 | 75 |
| 4 | 25 | 5 | 95 |
| 5 | 27 | 90 | 10 |

### <MS Conditions>

Mass Spectrometer: LCMS-8030 (manufactured by Shimadzu Corporation)
Measurement program: LC/MS/MS Method Package for Lipid Mediators Ver. 1

### (Manufactured by Shimadzu Corporation)

Nebulizer gas flow rate: 3 L/min
Drying gas flow rate: 15 L/min
DL temperature: 250° C.
Heat block temperature: 400° C.
Ionization mode: ESI+/−
Internal standard solution: Their composition was as indicated in Table 2.

TABLE 2
Compositions of internal standard solutions and corresponding lipid
mediators

| | Concentration | Corresponding lipid |
|---|---|---|
| Name of substance | (ng/ml) | mediator |
| tetranor-PGEM-d₆ | 200 | — |
| 6-keto PGF_{1α}-d₄ | 2000 | — |
| TXB₂-d₄ | 200 | — |
| PGF_{2α}-d₄ | 200 | 8-isoPGE₂ |
| PGE₂-d₄ | 200 | PGE₂ |
| PGD₂-d₄ | 200 | PGD₂ |
| LTC₄-d₅ | 200 | — |
| LTB₄-d₄ | 200 | 12-HEPE, 20-HETE, |
| | | 15-HEPE, 13-HODE |
| 5(S) HETE-d₈ | 1000 | — |
| 12(S) HETE-d₈ | 500 | 12-HETE, 14, 15-EET, |
| | | 14-HDoHE, 8-HDoHE, |
| | | 15-HEDE |
| 15(S) HETE-d₈ | 200 | 15-HETE, 16-HDoHE |
| PAF C16-d₄ | 200 | — |
| Oleoyl Ethaolamide-d₄ | 40 | 15-OxoEDE |

(2) Data processing

According to the above measurement program, the detected lipid mediators (133 types) were divided into 13 groups based on their physical properties, and internal standard substances were set for the respective groups (see Table 2). A quantification error or the like generated during lipid extraction was corrected by dividing the peak area value calculated from the chromatogram of each of the lipid mediators by the peak area value of the corresponding internal standard substance described above using LabSolutions (Version 5.80, manufactured by Shimadzu Corporation). The concentration of each of the lipid mediators (vertical axis in FIGS. 10 to 21) is shown as a value obtained by dividing the peak area value of the lipid mediator by the peak area value of the internal standard substance.

### (3) Results

As will be described below, the amounts of 15 types produced, among the detected lipid mediators (133 types), were observed to be significantly large, or to tend to be large, in the OVA-administered groups (that is, AR model mice in which mast cells and eosinophils were activated in the nasal cavities).

### A. Amount of Lipid Mediator Produced at Downstream of COX

As shown in FIG. 10, prostaglandin D2 (sometimes abbreviated as “PGD₂” herein) and prostaglandin E2 (sometimes abbreviated as “PGE₂” herein), which are lipid mediators derived from arachidonic acid (sometimes abbreviated as “AA” herein) and produced at the downstream of cyclooxygenase (COX), were detected in the nasal lavage fluids of the OVA-administered groups. The amount of PGD₂ produced was significantly larger in the 10-time OVA-administered group than in the 10-time solvent-administered group and the LPS-administered group (FIG. 10A). In addition, the amount of PGE₂ produced was significantly larger in the 10-time OVA-administered group than in the 10-time solvent-administered group, the 5-time OVA-administered group, and the LPS-administered group (FIG. 10B).

### B. Amount of Lipid Mediator Produced at Downstream of LOX

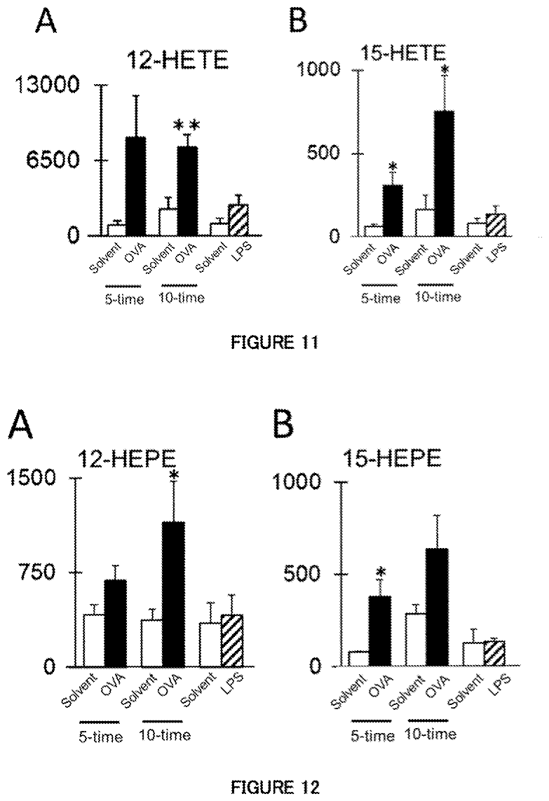

As shown in FIG. 11, 12-hydroxyeicosatetraenoic acid (sometimes abbreviated as “HETE” herein) and 15-HETE, which are lipid mediators derived from AA and produced at the downstream of 12/15-lipoxygenase (LOX), were detected in the nasal lavage fluids of the OVA-administered groups. The amount of 12-HETE produced was significantly larger in the 10-time OVA-administered group than in the 10-time solvent-administered group (FIG. 11A). Also, the amount of 15-HETE produced was significantly larger in the 5-time OVA-administered group and the 10-time OVA-administered group than in the 5-time solvent-administered group and the 10-time solvent-administered group, respectively (FIG. 11B).

As shown in FIG. 12, 12-hydroxy eicosapentaenoic acid (sometimes abbreviated as “HEPE” herein) and 15-HEPE, which are lipid mediators derived from eicosapentaenoic acid (sometimes abbreviated as “EPA” herein) and produced at the downstream of 12/15-LOX, were detected in the nasal lavage fluids of the OVA-administered groups. The amount of 12-HEPE produced was significantly larger in the 10-time OVA-administered group than in the 10-time solvent-administered group (FIG. 12A). Also, the amount of 15-HEPE produced was significantly larger in the 5-time OVA-administered group than in the 5-time solvent-administered group (FIG. 12B).

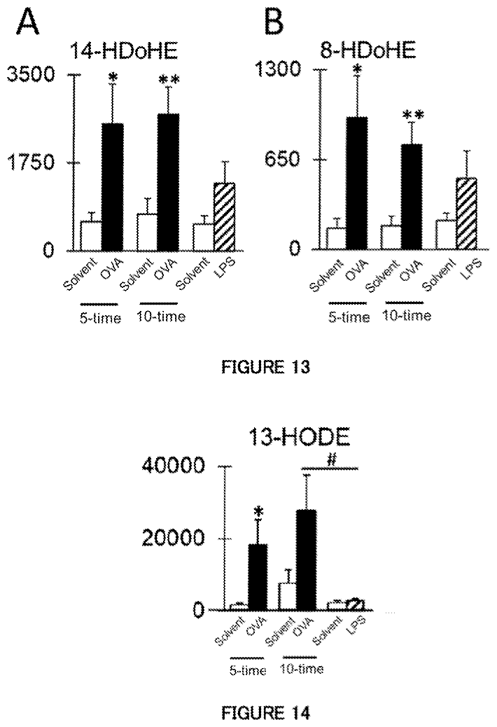

As shown in FIG. 13, 14-hydroxy docosahexaenoic acid (sometimes abbreviated as “HDoHE” herein), which is a lipid mediator derived from docosahexaenoic acid (and sometimes abbreviated as “DHA” herein), and 8-HDoHE, which is a lipid mediator produced at the downstream of 5-LOX, were detected in the nasal lavage fluids of the OVA-administered groups. The amount of 14-HDoHE produced was significantly larger in the 5-time OVA-administered group and the 10-time OVA-administered group than in the 5-time solvent-administered group and the 10-time solvent-administered group, respectively (FIG. 13A). Also, the amount of 8-HDoHE produced was significantly larger in the 5-time OVA-administered group and the 10-time OVA-administered group than in the 5-time solvent-administered group and the 10-time solvent-administered group, respectively (FIG. 13B).

As shown in FIG. 14, 13-hydroxy octadecadienoic acid (sometimes abbreviated as “HODE” herein), which is a lipid mediator derived from linolenic acid (α-linolenic acid, sometimes abbreviated as “LA” herein) and produced at the downstream of 12/15-LOX, was detected in the nasal lavage fluids of the OVA-administered groups. The amount of 13-HODE produced was significantly larger in the 5-time OVA-administered group than in the 5-time solvent-administered group. Also, it was significantly larger in the 10-time OVA-administered group than in the LPS-administered group.

### C. Amount of Lipid Mediator Produced Under OX

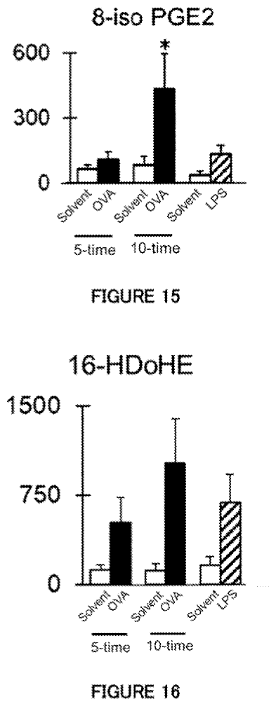

As shown in FIG. 15, 8-iso prostaglandin E2 (sometimes abbreviated as “isoPGE₂” herein), which is a lipid mediator derived from AA and produced at the downstream of enzyme-independent oxidation (OX), was detected in the nasal lavage fluids of the OVA-administered groups. The amount of 8-isoPGE₂ produced was significantly larger in the 10-time OVA-administered group than in the 10-time solvent-administered group.

As shown in FIG. 16, 16-HDoHE, which is a lipid mediator derived from DHA and produced under OX, was detected in the nasal lavage fluids of the OVA-administered groups. The amount of 16-HDoHE produced was observed to tend to be larger in the OVA-administered groups than in the solvent-administered groups.

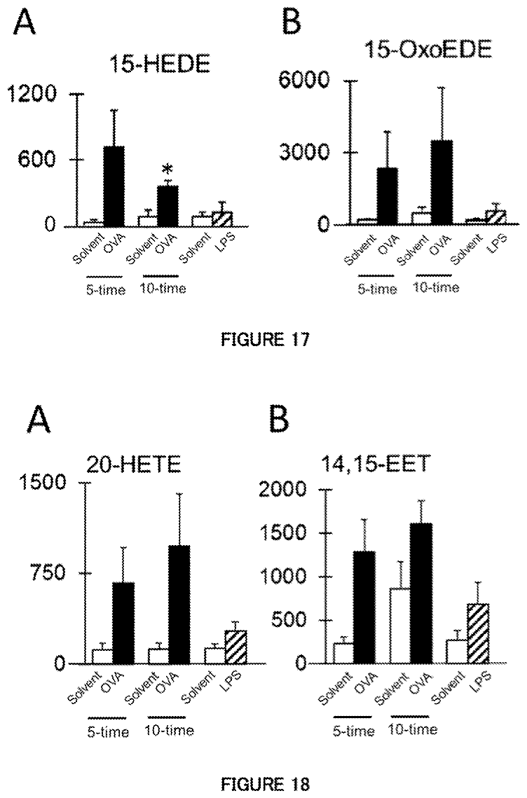

As shown in FIG. 17, 15-hydroxy eicosadienoic acid (sometimes abbreviated as “HEDE” herein) and 15-oxoeicosadienoic acid (sometimes abbreviated as OxoEDE herein), which are lipid mediators derived from eicosadienoic acid (sometimes abbreviated as “EDA” herein) and produced under OX, were detected in the nasal lavage fluids of the OVA-administered groups. The amount of 15-HEDE produced was significantly larger in the 10-time OVA-administered group than in the 10-time solvent-administered group. The amount of 15-OxoEDE produced was observed to tend to be larger in the OVA-administered groups than in the solvent-administered groups.

### D. Amount of Lipid Mediator Produced at Downstream of CYP

As shown in FIG. 18, 20-HETE and 14,15-epoxy eicosatrienoic acid (sometimes abbreviated as “EET” herein), which are lipid mediators derived from AA and produced at the downstream of cytochrome p450 (CYP), were detected in the nasal lavage fluids of the OVA-administered groups. The amount of 20-HETE produced was significantly larger in the 10-time OVA-administered group than in the 10-time solvent-administered group (FIG. 18A). Also, the amount of 14,15-EET produced was significantly larger in the 10-time OVA-administered group than in the 10-time solvent-administered group (FIG. 18B).

### Example 5: Lipid Mediator in Urine of Allergic Rhinitis and Upper Respiratory Inflammation Model Mice

### (1) Measurement of Lipid Mediator

### A. Preparation of Urine

The amounts of lipid mediators produced at the downstream of fatty acid metabolizing enzymes (COX, LOX and CYP) and under OX were inspected using the AR model mice (5-time OVA-administered group and 10-time OVA-administered group) and control groups (5-time solvent-administered group and 10-time solvent-administered group) (10 mice in each of the groups) prepared in Example 1. Urine was collected from the mice in each of the groups. Specifically, in the collection of urine from the 5-time OVA-administered group and the 5-time solvent-administered group, the mice were placed in a metabolic cage after the 4th administration of OVA or the solvent, and urine for 24 hours before the fifth administration was collected. Also, in the collection of urine from the 10-time OVA-administered group and the 10-time solvent-administered group, the mice were placed in a metabolic cage after the tenth administration of OVA or the solvent, and urine for 24 hours after the tenth administration was collected.

B. Lipid Extraction from Urine

The urine prepared in the above item A was centrifuged at 1000 rpm and 4° C. for 10 minutes. A sample lipid solution was prepared in the same manner as described in Example 4 (1) B except the sample solution was prepared by adding 10 μL of 0.05% formic acid water, 50 μL of the internal standard solution and 850 μL of deionized water to 100 μL of the supernatant.

### C. Measurement of Lipid Concentration by Liquid Chromatography-Mass Spectrometry

The lipid concentration was measured in the same manner as described in Example 4 (1) C.

### (2) Data Processing

Data processing was carried out in the same manner as described in Example 4 (2).

### (3) Results

### A. Amount of Lipid Mediator Produced at Downstream of COX

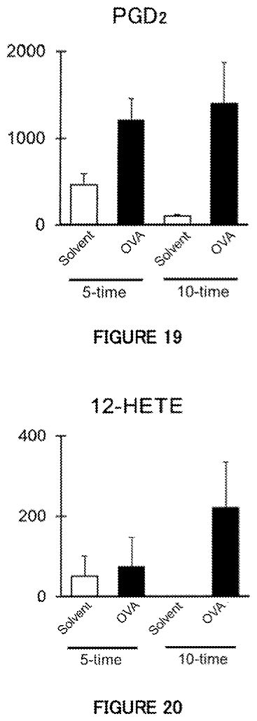

As shown in FIG. 19, PGD₂, which is a lipid mediator derived from AA and produced at the downstream of COX, was detected in the urine of the OVA-administered group. The amount of PGD₂ was observed to tend to be larger in the 5-time OVA-administered group and the 10-time OVA-administered group than in the solvent-administered groups.

### B. Amount of Lipid Mediator Produced at Downstream of LOX

As shown in FIG. 20, 12-HETE, which is a lipid mediator derived from AA and produced at the downstream of 12/15-LOX, was detected in the urine of the OVA-administered groups. The amount of 12-HETE was observed to tend to be larger in the 5-time OVA-administered group and the 10-time OVA-administered group than in the solvent-administered groups.

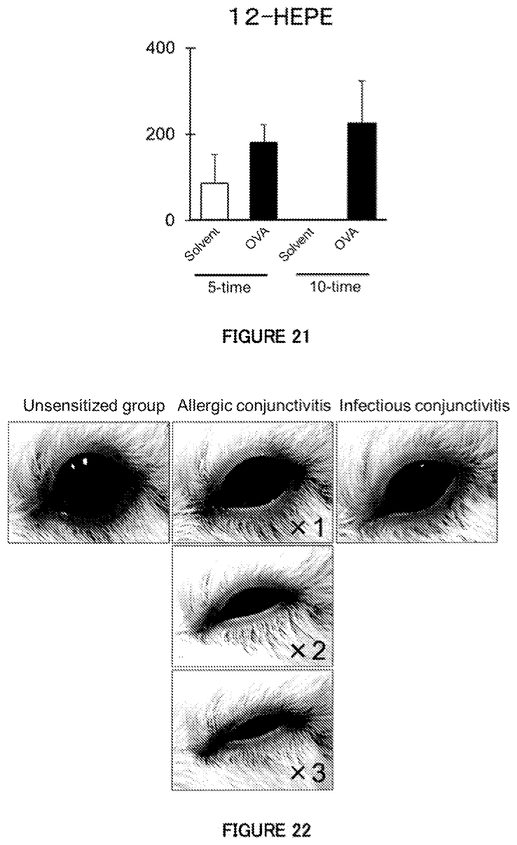

As shown in FIG. 21, 12-HEPE, which is a lipid mediator derived from EPA and produced at the downstream of 12/15-LOX, was detected in the urine of the OVA-administered groups. The amount of 12-HEPE was observed to tend to be larger in the 5-time OVA-administered group and the 10-time OVA-administered group than in the solvent-administered groups.

### Example 6: Preparation of Allergic Conjunctivitis Disease Model Guinea Pigs

### (1) Preparation of Allergic Conjunctivitis Model Guinea Pigs

To Kwl:Hartley guinea pigs (male, 7 to 11 weeks old) (3 guinea pigs), 100 μg of ovalbumin (manufactured by Sigma, sometimes abbreviated as “OVA” herein) dissolved and diluted with 10 μL of physiological saline (manufactured by Otsuka Pharmaceutical Co., Ltd.) and 1 mg of aluminum potassium sulfate (manufactured by Sigma-Aldrich Co. LLC.) were intraperitoneally administered simultaneously, and, after two weeks, 100 μg of OVA dissolved and diluted with 10 μL of physiological saline and 1 mg of aluminum potassium sulfate were intraperitoneally administered again for sensitization. Two weeks after the second sensitization, OVA (250 μg/10 μL) was administered by instillation once every other day for 11 days (6 times, in total) for stimulation, thereby preparing allergic conjunctivitis model guinea pigs.

### (2) Preparation of Infectious Conjunctivitis Model Guinea Pigs

Lipopolysaccharide (manufactured by Sigma, sometimes abbreviated as “LPS” herein) (5 μg/5 μL) was intraconjunctivally administered twice to 3 Kwl:Hartley guinea pigs (male, 7 to 11 weeks old), thereby preparing infectious conjunctivitis model guinea pigs. Hereinafter, the guinea pigs were used for testing 12 hours after the administration of LPS, unless otherwise specified.

### (3) Control Group (Solvent-Administered Group)

As a control group of allergic conjunctivitis, physiological saline (5 μL) was administered by instillation, in the amount and at the frequency employed in the above item (1), to 3 Kwl:Hartley guinea pigs (male, 7 to 11 weeks old) (solvent-administered group 1). As a control group of infectious conjunctivitis, physiological saline (5 μL) was administered by instillation, in the amount and at the frequency employed in the above item (2), to 3 Kwl:Hartley guinea pigs (male, 7 to 11 weeks old) (solvent-administered group 2).

### (4) Control Group (Unsensitized Group)

As a control group, Kwl:Hartley guinea pigs (male, 7 to 11 weeks old) were used (unsensitized group).

### Example 7: Evaluation of Symptoms of Allergic Conjunctivitis and Infectious, Conjunctivitis

### (1) Observation of Swelling of Conjunctiva

### A. Method

Swelling of the conjunctivae of the allergic conjunctivitis model guinea pigs (guinea pigs simulated with OVA once, twice or three times after two sensitizations with OVA), infectious conjunctivitis model guinea pigs and unsensitized group prepared in Example 6, was observed. The respective allergic conjunctivitis models were observed for the conjunctivae 30 minutes after the final stimulation with OVA, and the infectious conjunctivitis model guinea pigs were observed for the conjunctivae 10 hours after the administration of LPS.

### B. Results

The results were as shown in FIG. 22. Swelling of the conjunctivae was confirmed in both of the allergic conjunctivitis and infectious conjunctivitis model guinea pigs, as compared with the unsensitized group.

### (2) Evaluation of Vasopermeability

### A. Method

The vasopermeability, water content and thickness of the conjunctivae of the allergic conjunctivitis model guinea pigs (3 guinea pigs) and infectious conjunctivitis model guinea pigs (3 guinea pigs), and solvent-administered group 1 (3 guinea pigs) and solvent-administered group 2 (3 guinea pigs) as the control groups, which were prepared in Example 6. The vasopermeability was measured according to the Miles assay using Evans Blue (manufactured by Sigma). The water content was measured using a Schirmer test paper (manufactured by AYUMI Pharmaceutical Corporation). The thickness of the conjunctivae was measured using a caliper.

### B. Results

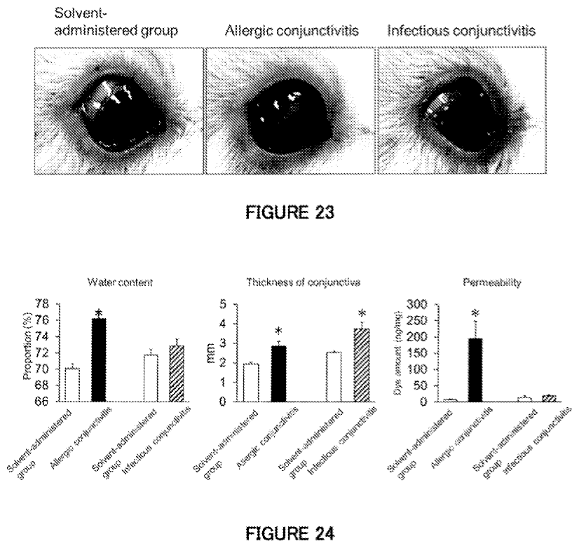

The results were as shown in FIGS. 23 and 24. The allergic conjunctivitis model were confirmed to show an increase in water content and a rise in vasopermeability as compared with the infectious conjunctivitis model or the solvent-administered group 1.

### (3) Histopathological Evaluation of Allergic Conjunctivitis and Infectious Conjunctivitis

### A. Method

Histopathological evaluation was performed on the conjunctivae of the allergic conjunctivitis model guinea pigs and infectious conjunctivitis model guinea pigs prepared in Example 6. Specifically, the method described in Example 3 was used.

### B. Results

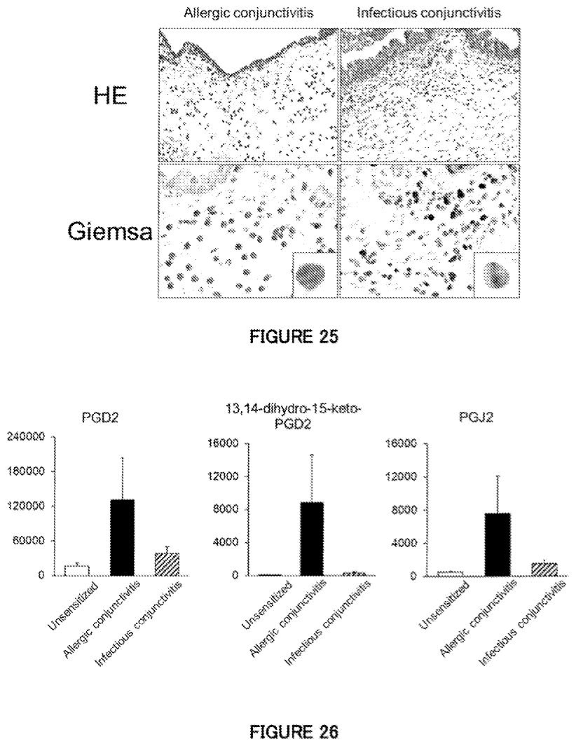

The results were as shown in FIG. 25. Infiltration of immunocytes according to each of the pathological conditions of the allergic conjunctivitis models and the infectious conjunctivitis models was observed.

### Example 8: Analysis of Lipid Mediator in Lacrimal Fluid and Conjunctival Sac Lavage Fluid of Allergic Conjunctivitis and Infectious Conjunctivitis Model Guinea Pigs

### (1) Measurement of Lipid Mediator

### A. Preparation of Lacrimal Fluid and Conjunctival Sac Lavage Fluid

The amounts of lipid mediators produced at the downstream of fatty acid metabolizing enzymes (COX, 5-LO, 12/15-LO and CYP) were inspected using the conjunctivae of the allergic conjunctivitis model guinea pigs (3 guinea pigs), infectious conjunctivitis model guinea pigs (3 guinea pigs) and unsensitized group (3 guinea pigs) prepared in Example 6. Lacrimal fluids and conjunctival sac lavage fluids were prepared by reverse phase solid-phase extraction.

B. Lipid Extraction from Lacrimal Fluid and Conjunctival Sac Lavage Fluid

Lipid extraction from the lacrimal fluids and conjunctival sac lavage fluids prepared in the above item A was performed according to the description in Example 4 (1) B.

### C. Measurement of Lipid Concentration by Liquid Chromatography-Mass Spectrometry

The lipid concentrations of the sample solutions prepared in the above item B were measured according to the description in Example 4 (1) C.

### (2) Data Processing

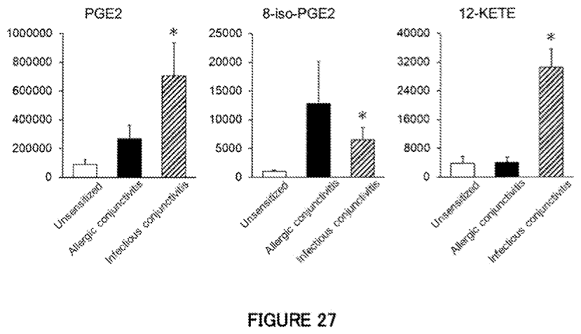

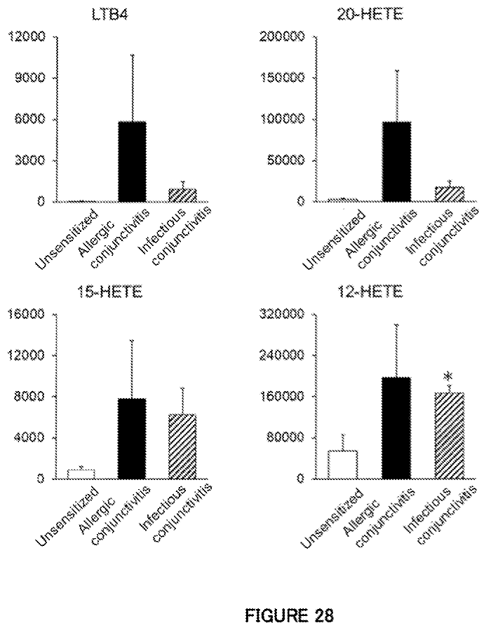

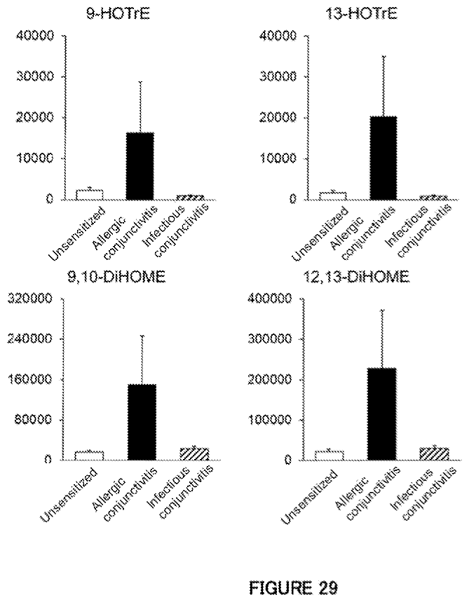

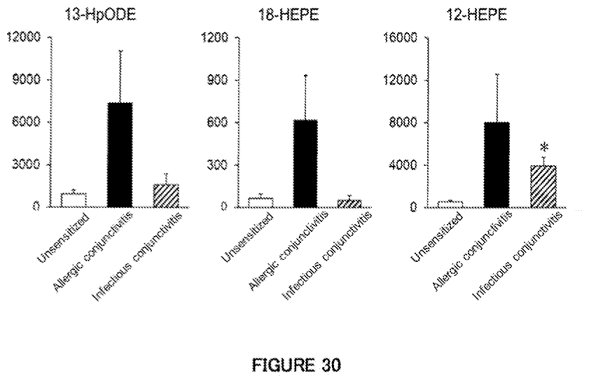

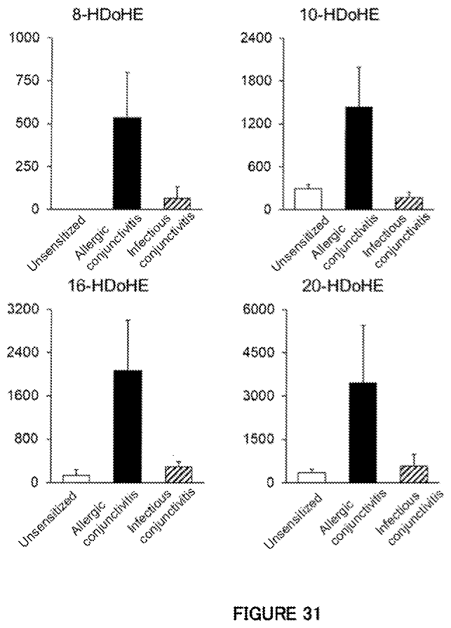

The lipid mediator concentrations of the lipid mediators detected in the above item (1) (vertical axes in FIGS. 26 to 31) were determined according to the description in Example 4 (2).

### (3) Results

The results were as shown in FIGS. 26 to 31. Among the detected lipid mediators, the amounts of PGD₂, 13,14-dihydro-15-keto-PGD₂, PGJ₂, LTB₄, 20-HETE, 15-HETE, 12-HETE, 9-HOTrE, 13-HOTrE, 9,10-DiHOME, 12,13-DiHOME, 13-HpODE, 18-HEPE, 12-HEPE, 8-HDoHE, 10-HDoHE, 16-HDoHE and 20-HDoHE produced were observed to be significantly large, or to tend to be large, in the allergic conjunctivitis model.

1. A method for detecting an allergic disease or an activation state of mast cells and/or eosinophils in the nasal cavities or eyes, comprising the step of measuring the concentration of a lipid metabolite in a biological sample of a subject.

2. The method for detection according to claim 1, wherein, when the concentration of the lipid metabolite in the biological sample of the subject is above the concentration of the lipid metabolite in a biological sample of a healthy subject, it is indicated that the subject suffers from an allergic disease or that mast cells and/or eosinophils are activated in the nasal cavities or eyes of the subject.

3. A method for determining a therapeutic effect on an allergic disease, comprising the step of measuring the concentration of a lipid metabolite m a biological sample of a subject.

4. The method for determination according to claim 3, wherein, when the concentration of the lipid metabolite in the biological sample of the subject is below the concentration of the lipid metabolite in the biological sample of the subject before treatment or the concentration of the lipid metabolite in a biological sample of a subject suffering from an allergic disease, it is indicated that the treatment has a therapeutic effect.

5. The method for detection according to claim 1, wherein the allergic disease is allergic rhinitis or allergic conjunctivitis.

6. The method for determination according to claim 3, wherein the allergic disease is allergic rhinitis, and wherein the treatment of allergic rhinitis is drug therapy, immunotherapy or surgical treatment.

7. The method for detection according to claim 5, wherein the allergic rhinitis is seasonal allergic rhinitis or perennial allergic rhinitis.

8. The method for detection according to claim 1, wherein the biological sample is a body fluid noninvasively collected from the subject.

9. The method for detection according to claim 1, wherein the lipid metabolite is one or more lipid metabolite(s) selected from the group consisting of an arachidonic acid metabolite, an eicosapentaenoic acid metabolite, a docosahexaenoic acid metabolite, a linolenic acid metabolite and an eicosadienoic acid metabolite.

10. The method for detection according to claim 1, wherein the lipid metabolite is one or more lipid metabolite(s) selected from the group consisting of PGD₂, PGE₂, 12-HETE, 15-HETE, 12-HEPE, 15-HEPE, 14-HDOHE, 8-HDOHE, 13-HODE, 8-isoPGE₂, 16-HDoHE, 15-HEDE, 15-OxoEDE, 20-HETE, 14,15-EET, 13,14-dihydro-15-keto-PGD₂, PGJ₂, LTB₄, 9-HOTrE, 13-HOTrE, 9,10-DiHOME, 12,13-DiHOME, 13-HpODE, 18-HEPE, 10-HDoHE and 20-HDoHE.

11. The method for detection according to claim 1, wherein the concentration of the lipid metabolite is measured by mass spectrometry.

12-16. (canceled)

17. A method for screening for a therapeutic drug for an allergic disease, comprising the steps of administering a candidate drug for a therapeutic drug for an allergic disease to a subject; and measuring the concentration of a lipid metabolite in a biological sample of the subject.

18. The method for screening according to claim 17, wherein, when the concentration of the lipid metabolite in the biological sample of the subject after the administration of the candidate drug is below the concentration of the lipid metabolite in the biological sample of the subject before the administration of the candidate drug or the concentration of the lipid metabolite in a biological sample of a subject suffering from the allergic disease, it is indicated that the therapeutic drug has a therapeutic effect.

19. A method for identifying, in a lipid metabolite in a biological sample, an allergic disease marker or an activation marker for mast cells and/or eosinophils in the nasal cavities or eyes, comprising the steps of measuring the concentration of a lipid metabolite in a biological sample of a subject suffering from an allergic disease and the concentration of the lipid metabolite in a biological sample of a healthy subject; and comparing the measured two concentrations.

20. The method for identification according to claim 19, wherein, when the concentration of the lipid metabolite in the biological sample of the subject suffering from the allergic disease is above the concentration of the lipid metabolite in the biological sample of the healthy subject, it is indicated that the lipid metabolite serves as an allergic disease marker or an activation marker for mast cells and/or eosinophils in the nasal cavities or eyes.

21. The method for screening according to claim 17, wherein the allergic disease is allergic rhinitis or allergic conjunctivitis.
